(19) United States
(12) Patent Application Publication
(10) Pub. No.: US 2026/0354379 A1
(43) Pub. Date: Nov. 12, 2026

(54) SOLID FORM OF A HETEROCYCLIC AMIDE DERIVATIVE

(71) Applicant: SYNGENTA CROP PROTECTION AG, Basel (CH)

(72) Inventors: John HONE, Bracknell, Berkshire (GB); Mathieu MARMIER, Monthey (CH); Andrei BADOIU, Monthey (CH)

(73) Assignee: SYNGENTA CROP PROTECTION AG, Basel (CH)

(21) Appl. No.: 18/997,688

(22) PCT Filed: Jul. 14, 2023
(86) PCT No.: PCT/EP2023/069636

§ 371 Date: Jan. 22, 2025

(30) Foreign Application Priority Data
Jul. 22, 2022 (EP) 22186568.6

(51) Int. Cl. A01N 43/56; A01P 3/00; C07D 231/14

(52) U.S. Cl. CPC A01N 43/56; A01P 3/00; C07D 231/14

(57) ABSTRACT

The present invention relates to a crystalline polymorph of the compound of formula (I), which has a powder X-ray diffraction pattern comprising at least three 2θ angle values selected from the group consisting of 7.2±0.2°, 10.8±0.2°, 13.8±0.2°, 14.4±0.2°, 14.8±0.2°, 15.9±0.2°, 16.4±0.2°, 19.9±0.2°, 20.7±0.2°, 22.2±0.2°, 22.6±0.2°, 23.9±0.2°, 24.6±0.2°, 26.0±0.2°, 26.3±0.2°, 27.5±0.2°, 30.0±0.2°, 32.0±0.2°, and 32.7±0.2°.

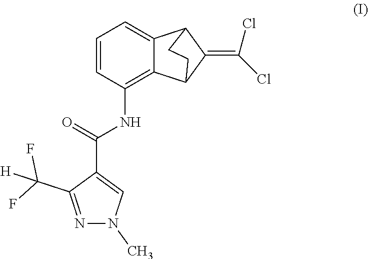 [SMILES: Cn1cc(C(=O)Nc2cccc3c2C2CCC3C2=C(Cl)Cl)c(C(F)F)n1]

This invention relates to a solid form of a heterocyclic amide derivative, a process to prepare said solid form, to a composition comprising said solid form, and to a method of its use as fungicide.

WO 2007/048556 discloses that certain heterocyclic amide derivatives have microbicidal activity, such as fungicidal activity. In particular, a compound of formula I, 3-difluoromethyl-1-methyl-1H-pyrazole-4-carboxylic acid(9-dichloromethylene-1,2,3,4-tetrahydro-1,4-methano-naphthalen-5-yl)-amide is disclosed:

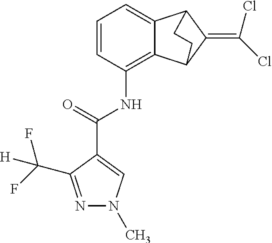 [SMILES: Cn1cc(C(=O)Nc2cccc3c2C2CCC3C2=C(Cl)Cl)c(C(F)F)n1]

A new solid form of this compound and its preparation have now been discovered. Accordingly, the present invention relates to a novel crystalline form of a heterocyclic amide derivative, said derivative being a crystalline polymorph of the compound of formula I:

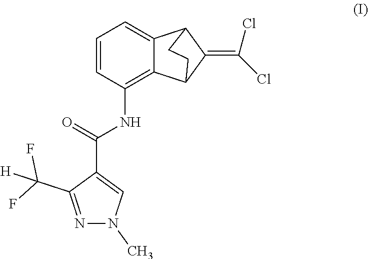 [SMILES: Cn1cc(C(=O)Nc2cccc3c2C2CCC3C2=C(Cl)Cl)c(C(F)F)n1]

The crystalline polymorph according to the present invention presents an optimized handling and processing, especially on manufacturing scale. More particularly, the crystalline polymorph can guarantee a significant improvement in reducing filtration times.

More particularly, the crystalline polymorph of formula I may be characterised by a powder X-ray diffraction pattern expressed in terms of 2θ angles and/or d spacings.

The crystalline polymorph of formula I can have a powder X-ray diffraction pattern comprising at least three 2θ angle values, preferably at least six 2θ angle values, preferably at least eight 2θ angle values, and more preferably at least ten 2θ angle values, selected from the group consisting of 7.2±0.2°, 10.8±0.2°, 13.8±0.2°, 14.4±0.2°, 14.8±0.2°, 15.9±0.2°, 16.4±0.2°, 19.9±0.2°, 20.7±0.2°, 22.2±0.2°, 22.6±0.2°, 23.9±0.2°, 24.6±0.2°, 26.0±0.2°, 26.3±0.2°, 27.5±0.2°, 30.0±0.2°, 32.0±0.2°, and 32.7±0.2°.

In a preferred embodiment, the crystalline polymorph of formula I can have a powder X-ray diffraction pattern comprising at least three 2θ angle values, preferably at least six 2θ angle values, preferably at least eight 2θ angle values, and more preferably at least ten 2θ angle values, selected from the group consisting of 7.2±0.2°, 10.8±0.2°, 13.8±0.2°, 14.4±0.2°, 14.8±0.2°, 16.4±0.2°, 20.7±0.2°, 22.2±0.2°, 24.6±0.2°, 26.0±0.2°, 27.5±0.2°, 30.0±0.2°, 32.0±0.2°, and 32.7±0.2°.

More particularly, the crystalline polymorph of formula I can have a powder X-ray diffraction pattern can comprise at least the following 2θ angle values: 7.2±0.2°, 10.8±0.2°, 13.8±0.2°, 14.4±0.2°, 14.8±0.2°, 16.4±0.2°, 20.7±0.2°, 22.2±0.2°, 24.6±0.2°, and 26.0±0.2°; preferably at least the following 2θ angle values: of 7.2±0.2°, 10.8±0.2°, 13.8±0.2°, 14.4±0.2°, 14.8±0.2°, 16.4±0.2°, 20.7±0.2°, 22.2±0.2°, 24.6±0.2°, 26.0±0.2°, 27.5±0.2°, 30.0±0.2°, 32.0±0.2° and 32.7±0.2°; and more preferably at least the following 2θ angle values: 7.2±0.2°, 10.8±0.2°, 13.8±0.2°, 14.4±0.2°, 14.8±0.2°, 15.9±0.2°, 16.4±0.2°, 19.9±0.2°, 20.7±0.2°, 22.2±0.2°, 22.6±0.2°, 23.9±0.2°, 24.6±0.2°, 26.0±0.2°, 26.3±0.2°, 27.5±0.2°, 30.0±0.2°, 32.0±0.2°, and 32.7±0.2°.

The d spacing values are calculated using an average wavelength of 1.540598 Å, and are given in Table 1 for the above-mentioned 2θ angle values.

TABLE 1

| 2θ/° | d/Å |
|---|---|
| 7.2 ± 0.2 | 12.24 ± 0.34 |
| 10.8 ± 0.2 | 8.19 ± 0.15 |
| 13.8 ± 0.2 | 6.42 ± 0.09 |
| 14.4 ± 0.2 | 6.13 ± 0.08 |
| 14.8 ± 0.2 | 5.99 ± 0.08 |
| 15.9 ± 0.2 | 5.56 ± 0.07 |
| 16.4 ± 0.2 | 5.40 ± 0.07 |
| 19.9 ± 0.2 | 4.47 ± 0.04 |
| 20.7 ± 0.2 | 4.30 ± 0.04 |
| 22.2 ± 0.2 | 4.01 ± 0.04 |
| 22.6 ± 0.2 | 3.92 ± 0.03 |
| 23.9 ± 0.2 | 3.73 ± 0.03 |
| 24.6 ± 0.2 | 3.61 ± 0.03 |
| 26.0 ± 0.2 | 3.43 ± 0.03 |
| 26.3 ± 0.2 | 3.38 ± 0.03 |
| 27.5 ± 0.2 | 3.24 ± 0.02 |
| 30.0 ± 0.2 | 2.97 ± 0.02 |
| 32.0 ± 0.2 | 2.80 ± 0.02 |
| 32.7 ± 0.2 | 2.73 ± 0.02 |

The crystalline polymorph of the invention may be characterised by the unit cell parameters of its single crystal as shown in Table 2.

TABLE 2

| Class | Triclinic |
|---|---|
| Space Group | P 1̅ |
| Cell Lengths (Å) | a = 8.0 ± 0.1; b = 14.0 ± 0.1; c = 17.0 ± 0.1 |
| Cell Angles (°) | α = 70.2 ± 0.2; β = 84.1 ± 0.2; γ = 74.4 ± 0.2 |
| Unit Cell Volume (Å³) | 1724 ± 20 |

In Table 2, a, b, c are the lengths of the edges of the unit cell and α, β, γ are the angles of the unit cell.

In a particular embodiment of the present invention, the crystalline polymorph can have the following lattice parameters: a=8.0±0.1 Å, b=14.0 Å±0.1 Å, c=17.0 Å±0.1 Å, α=70.2±0.2°, β=84.1±0.2°, γ=74.4±0.2°, and volume=1724=20 Å³.

In another embodiment, the crystalline polymorph according to the invention can have a melting point ranging from 153 to 156 °C (peak position), and preferably ranging from 154 to 155 °C (peak position). This melting point is obtained using Differential Scanning calorimetry (DSC) with a heating rate of 10 °C/minute.

In the context of the present invention, a polymorph is a particular crystal form of a chemical compound that can exist in more than one crystal form in the solid state. A crystal form of a compound contains the constituent molecules arranged in orderly repeating patterns extending in all three spatial dimensions (in contrast, an amorphous solid form has no long-range order in the position of molecules). Different polymorphs of a compound have different arrangements of atoms and or molecules in their crystal structure. When the compound is a biologically active compound, such as a fungicide, the difference in crystal structures can lead to different polymorphs having different chemical, physical and biological properties. Properties which may be affected include crystal shape, density, hardness, colour, chemical stability, melting point, hydroscopicity, suspensibility, dissolution rate and biological availability. As such, a specific polymorph may have properties which make it more advantageous in a particular use relative to another polymorph of the same compound: in particular, the physical, chemical and biological properties listed above can have a significant effect on the development of production methods, especially at manufacturing scale, and on formulations; on the ease with which a compound can be combined in a formulation with other active ingredients and formulation components; and/or on the quality and efficacy of plant treatment agents, such as fungicides. It is noted that predicting whether the solid state of a compound may be present as more than one polymorph is not possible and nor is it possible to predict the properties of any of these crystal forms.

Assaying the solid phase for the presence of crystals may be carried out by conventional methods known in the art. For example, it is convenient and routine to use powder X-ray diffraction techniques. Other techniques which may be used include differential scanning calorimetry (DSC), near infra-red spectroscopy, mid infra-red spectroscopy, nuclear magnetic resonance (NMR), gas chromatography or high-performance liquid chromatography (HPLC). Single crystal X-ray diffraction is especially useful in identifying crystal structures.

Another object of the present invention relates to a method of preparing the crystalline polymorph of the compound of formula I, comprising the following steps:
i. mixing the technical compound of formula I with an organic solvent, at a temperature of at least 30 °C, and more preferably of at least 40 °C, in order to obtain a saturated solution of the technical compound of formula I,
ii. optionally, filtering the mixture obtained in step i,
iii. crystallizing the compound obtained in step ii at a temperature less than 30 °C, and preferably up to 25 °C, to obtain a suspension, and
iv. storing the suspension obtained in step iii, preferably for at least 2 hours, preferably for at least 4 hours, preferably for at least 10 hours, preferably for at least 15 days, and more preferably for at least 30 days, at a temperature less than 30 °C and preferably up to 25 °C, to obtain the crystalline polymorph of the compound of formula I.

The technical compound of formula I is a racemic mixture of the compound of formula I (3-difluoromethyl-1-methyl-1H-pyrazole-4-carboxylic acid (9-dichloromethylene-1,2,3,4-tetrahydro-1,4-methano-naphthalen-5-yl)-amide) with a purity of at least 98%, and more preferably of at least 99%, prepared as described in Example P11 of WO 2012/101139.

The organic solvent can be selected among xylene, isopropyl alcohol, chlorobenzene, acetone, methylcyclohexane, ethyl acetate, methanol, acetonitrile, and any mixture thereof.

In step i, the saturated solution means that no more solute (compound of formula I) can be dissolved in the solvent.

In step ii, the filtration step can help to remove all remaining solid particles, such as for example undissolved solids, crystals, and/or dust. This optional step can limit or prevent nucleation points during the preparation method.

The step iv can be advantageously performed under stirring, and preferably under constant stirring. The stirring can be a mild stirring, and more particularly a constant mild stirring. In the present invention, a mild stirring means a sufficient stirring to suspend the solids.

The step iv can be followed by a filtering step v of the stored suspension, in order to isolate the crystals generated in the step iv from their solvent(s) and use them as solid seeds.

The polymorphs of the invention may be applied in unchanged form but are more preferably incorporated into an agrochemical composition by conventional means. Accordingly, a further object according to the invention relates to an agrochemical composition comprising the crystalline polymorph as defined in the present invention, and at least one an agriculturally acceptable carrier or diluent. The agrochemical composition comprising the crystalline polymorph of the present invention can be used for the control of plant pathogenic fungi on a number of plant species.

Another object of the present invention relates to a method of preventing or controlling fungal infection on plants or plant propagation material comprising treating the plant or plant propagation material with said agrochemical composition, and preferably with a fungicidally effective amount of said agrochemical composition.

The term “plants” refers to all physical parts of a plant, including seeds, seedlings, saplings, roots, tubers, stems, stalks, foliage, and fruits.

The term “plant propagation material” is understood to denote all the generative parts of the plant, such as seeds, which can be used for the multiplication of the latter including vegetative plant material such as cuttings. There may be mentioned, as plant propagation material, seeds (in the strict sense), roots, fruits, tubers, bulbs, rhizomes, parts of plants. Germinated plants and young plants which are to be transplanted after germination or after emergence from the soil, may also be mentioned. These young plants may be protected before transplantation by a total or partial treatment by immersion. The plant propagation material can be treated with the composition of the invention before the material is sown or planted. Alternatively, the plant propagation material may be treated with the composition of the invention during sowing or planting. Additionally, the composition of the invention may be applied to the previously treated propagation material before or during its planting. The composition of the invention may be applied during the sowing of the seed. The composition may also be used to plant propagation material derived from plants grown in a green house and/or during transplantation.

More preferably the plant propagation material is plant seeds. The seed treatment can occur to an unsown seed, and the term “unsown seed” is meant to include seed at any period between the harvest of the seed and the sowing of the seed in the ground for the purpose of germination and growth of the plant. Treatment to an unsown seed is not meant to include those practices in which the composition is applied to the soil but would include any application practice that would target the seed during the sowing/planting process. The treated plant propagation material of the present invention can be treated in the same manner as conventional plant propagation material. The treated propagation material can be stored, handled, sown and tilled in the same manner as any other pesticide treated material.

The agrochemical composition of the invention can be used to control, for example, phytopathogenic fungi belonging to the following classes: Ascomycetes (e.g. Venturia, Podosphaera, Erysiphe, Monilinia, Mycosphaerella, Uncinula); Basidiomycetes (e.g. the genus Hemileia, Rhizoctonia, Phakopsora, Puccinia, Ustilago, Tilletia); Fungi imperfecti (also known as Deuteromycetes; e.g. Botrytis, Helminthosporium, Rhynchosporium, Fusarium, Septoria, Cercospora, Alternaria, Pyricularia and Pseudocercosporella); Oomycetes (e.g. Phytophthora, Peronospora, Pseudoperonospora, Albugo, Bremia, Pythium, Pseudosclerospora, Plasmopara).

The agrochemical composition of the present invention is suitable for controlling such disease on a number of plants and their propagation material including, but not limited to the following target crops: cereals (wheat, barley, rye, oats, maize (including field corn, pop corn and sweet corn), rice, sorghum and related crops); beet (sugar beet and fodder beet); leguminous plants (beans, lentils, peas, soybeans); oil plants (rape, mustard, sunflowers); cucumber plants (marrows, cucumbers, melons); fibre plants (cotton, flax, hemp, jute); vegetables (spinach, lettuce, asparagus, cabbages, carrots, eggplants, onions, pepper, tomatoes, potatoes, paprika, okra); plantation crops (bananas, fruit trees, rubber trees, tree nurseries), ornamentals (flowers, shrubs, broad-leaved trees and evergreens, such as conifers); as well as other plants such as vines, bushberries (such as blueberries), caneberries, cranberries, peppermint, rhubarb, spearmint, sugar cane and turf grasses including, but not limited to, cool-season turf grasses (for example, bluegrasses (Poa L.), such as Kentucky bluegrass (Poa pratensis L.), rough bluegrass (Poa trivialis L.), Canada bluegrass (Poa compressa L.) and annual bluegrass (Poa annua L.); bentgrasses (Agrostis L.), such as creeping bentgrass (Agrostis palustris Huds.), colonial bentgrass (Agrostis tenius Sibth.), velvet bentgrass (Agrostis canina L.) and redtop (Agrostis alba L.); fescues (Festuca L.), such as tall fescue (Festuca arundinacea Schreb.), meadow fescue (Festuca elatior L.) and fine fescues such as creeping red fescue (Festuca rubra L.), chewings fescue (Festuca rubra var. commutata Gaud.), sheep fescue (Festuca ovina L.) and hard fescue (Festuca longifolia); and ryegrasses (Lolium L.), such as perennial ryegrass (Lolium perenne L.) and annual (Italian) ryegrass (Lolium multiflorum Lam.)) and warm-season turf grasses (for example, Bermudagrasses (Cynodon L. C. Rich), including hybrid and common Bermudagrass; Zoysiagrasses (Zoysia Willd.), St. Augustinegrass (Stenotaphrum secundatum (Walt.) Kuntze); and centipedegrass (Eremochloa ophiuroides (Munro.) Hack.)).

In addition ‘crops’ are to be understood to include those crops that have been made tolerant to pests and pesticides, including herbicides or classes of herbicides, as a result of conventional methods of breeding or genetic engineering. Tolerance to e.g. herbicides means a reduced susceptibility to damage caused by a particular herbicide compared to conventional crop breeds. Crops can be modified or bred so as to be tolerant, for example, to HPPD inhibitors such as mesotrione or EPSPS inhibitors such as glyphosate.

In particular, the composition according to the invention is particularly effective against powdery mildews; rusts; leafspot species; early blights and molds; especially against Septoria, Puccinia, Erysiphe, Pyrenophora and Tapesia in cereals; Phakopsora in soybeans; Hemileia in coffee; Phragmidium in roses; Alternaria in potatoes, tomatoes and cucurbits; Sclerotinia in turf, vegetables, sunflower and oil seed rape; black rot, red fire, powdery mildew, grey mold and dead arm disease in vine; Botrytis cinerea in fruits; Monilinia spp. in fruits and Penicillium spp. in fruits.

The composition according to the invention is furthermore particularly effective against seedborne and soilborne diseases, such as Alternaria spp., Ascochyta spp., Botrytis cinerea, Cercospora spp., Claviceps purpurea, Cochliobolus sativus, Colletotrichum spp., Epicoccum spp., Fusarium graminearum, Fusarium moniliforme, Fusarium oxysporum, Fusarium proliferatum, Fusarium solani, Fusarium subglutinans, Gäumannomyces graminis, Helminthosporium spp., Microdochium nivale, Phoma spp., Pyrenophora graminea, Pyricularia oryzae, Rhizoctonia solani, Rhizoctonia cerealis, Sclerotinia spp., Septoria spp., Sphacelotheca reilliana, Tilletia spp., Typhula incarnata, Urocystis occulta, Ustilago spp. or Verticillium spp.; in particular against pathogens of cereals, such as wheat, barley, rye or oats; maize; rice; cotton; soybean; turf; sugarbeet; oil seed rape; potatoes; pulse crops, such as peas, lentils or chickpea; and sunflower.

The composition according to the invention is furthermore particularly effective against post harvest diseases such as Botrytis cinerea, Colletotrichum musae, Curvularia lunata, Fusarium semitecum, Geotrichum candidum, Monilinia fructicola, Monilinia fructigena, Monilinia laxa, Mucor piriformis, Penicilium italicum, Penicilium solitum, Penicillium digitatum or Penicillium expansum in particular against pathogens of fruits, such as pomefruits, for example apples and pears, stone fruits, for example peaches and plums, citrus, melons, papaya, kiwi, mango, berries, for example strawberries, avocados, pomegranates and bananas, and nuts.

The composition according to the invention is particularly useful for controlling the following diseases on the following crops: Alternaria species in fruit and vegetables; Ascochyta species in pulse crops; Botrytis cinerea in strawberries, tomatoes, sunflower, pulse crops, vegetables and grapes, such as Botrytis cinerea on grape; Cercospora arachidicola in peanuts; Cochliobolus sativus in cereals; Colletotrichum species in pulse crops; Erysiphe species in cereals; such as Erysiphe graminis on wheat and Erysiphe graminis on barley; Erysiphe cichoracearum and Sphaerotheca fuliginea in cucurbits; Fusarium species in cereals and maize; Gäumannomyces graminis in cereals and lawns; Helminthosporium species in maize, rice and potatoes; Hemileia vastatrix on coffee; Microdochium species in wheat and rye; Mycosphaerella fijiensis in banana; Phakopsora species in soybeans, such as Phakopsora pachyrizi in soybeans; Puccinia species in cereals, broadleaf crops and perennial plants; such as Puccinia recondita on wheat, Puccinia striiformis on wheat and Puccinia recondita on barley; Pseudocercosporella species in cereals, such as Pseudocercosporella herpotrichoides in wheat; Phragmidium mucronatum in roses; Podosphaera species in fruits; Pyrenophora species in barley, such as Pyrenophora teres on barley; Pyricularia oryzae in rice; Ramularia collo-cygni in barley; Rhizoctonia species in cotton, soybean, cereals, maize, potatoes, rice and lawns, such as Rhizoctonia solani on potato, rice, turf and cotton; Rhynchosporium secalis on barley, Rhynchosporium secalis on rye; Sclerotinia species in lawns, lettuce, vegetables and oil seed rape, such as Sclerotinia sclerotiorum on oilseed rape and Sclerotinia homeocarpa on turf; Septoria species in cereals, soybean and vegetables, such as Septoria tritici on wheat, Septoria nodorum on wheat and Septoria glycines on soybean; Sphacelotheca reilliana in maize; Tilletia species in cereals; Uncinula necator, Guignardia bidwellii and Phomopsis viticola in vines; Urocystis occulta in rye; Uromyces species in beans; Ustilago species in cereals and maize; Venturia species in fruits, such as Venturia inequalis on apple; Monilinia species on fruits; Penicillium species on citrus and apples.

The rate at which the agrochemical composition of the invention is applied will depend upon the particular type of fungus to be controlled, the degree of control required and the timing and method of application and can be readily determined by the person skilled in the art. In general, the composition of the invention can be applied at an application rate of between 0.005 kilograms/hectare (kg/ha) and about 5.0 kg/ha, based on the total amount of active fungicide in the composition. An application rate of between about 0.1 kg/ha and about 1.5 kg/ha is preferred, with an application rate of between about 0.3 kg/ha and 0.8 kg/ha being especially preferred.

In practice, the agrochemical composition comprising the polymorphs of the invention is applied as a formulation containing the various adjuvants and carriers known to or used in the industry. They may thus be formulated as granules, as wettable powders, as emulsifiable concentrates, as suspension concentrates (including oil dispersions), as powders or dusts, as flowables, as solutions, as suspensions or emulsions, suspo-emulsions or as controlled release forms such as microcapsules. Suitably, the agrochemical composition of the invention may be formulated as a suspension concentrate, a suspo-emulsion an emulsion concentrate or a wet granulation. These formulations are described in more detail below and may contain as little as about 0.5% to as much as about 95% or more by weight of the active ingredient in the form of the polymorph. The optimum amount will depend on formulation, application equipment and nature of the plant pathogenic fungi to be controlled.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain by weight about 5% to about 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient by weight may range from about 0.5% to about 95% of the concentrate.

Suspension concentrates are formulations in which finely divided solid particles of the active compound are stably suspended. The solid particles may be suspended in an aqueous solution or in an oil (as an oil dispersion). Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient by weight may range from about 0.5% to about 95% of the concentrate.

Granular formulations include both extrudates and relatively coarse particles and may be applied without dilution to the area in which control of plant pathogenic fungi is required or dispersed in a spray tank before application, for example. Typical carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations for use without dilution normally contain by weight about 5% to about 25% active ingredients which may include surface-active agents such as heavy aromatic naphthas, kerosene and other petroleum fractions, or vegetable oils; and/or stickers such as dextrins, glue or synthetic resins. When the granules are to be dispersed in a spray tank before application, the active ingredient content by weight may be increased up to 80%.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically from about 1 to about 50 microns in diameter. The enclosed liquid typically constitutes about 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre (and preferably from 1 to 2 millimetres) in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

Many of the formulations described above include wetting, dispersing or emulsifying agents. Examples are alkyl and alkylaryl sulphonates and sulphates and their salts, polyhydric alcohols; polyethoxylated alcohols, esters and fatty amines. These agents, when used, normally comprise from 0.1% to 40% by weight of the formulation.

Suitable agricultural adjuvants and carriers that are useful in formulating the composition of the invention in the formulation types described above are well known to those skilled in the art. Suitable examples of the different classes are found in the non-limiting list below.

Liquid carriers that can be employed include water and any solvents in which the polymorph has no or limited solubility e.g. toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, diacetonalcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulphonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, ethylene glycol, propylene glycol, glycerine, N-methyl-2-pyrrolidinone, and the like. Water is generally the carrier of choice for the dilution of concentrates.

Suitable solid carriers include talc, titanium dioxide, pyrophyllite clay, silica, attapulgite clay, kieselguhr, chalk, diatomaceous earth, lime, calcium carbonate, bentonite clay, fuller's earth, cotton seed hulls, wheat flour, soybean flour, pumice, wood flour, walnut shell flour, lignin and the like.

A broad range of surface-active agents are advantageously employed in both said liquid and solid compositions, especially those designed to be diluted with carrier before application. The surface-active agents can be anionic, cationic, non-ionic or polymeric in character and can be employed as emulsifying agents, wetting agents, suspending agents or for other purposes. Typical surface-active agents include salts of alkyl sulphates, such as diethanolammonium lauryl sulphate; alkylarylsulphonate salts, such as calcium dodecylbenzenesulphonate; alkylphenol-alkylene oxide addition products, such as nonylphenol-C.sub. 18 ethoxylate; alcohol-alkylene oxide addition products, such as tridecyl alcohol-C.sub. 16 ethoxylate; soaps, such as sodium stearate; alkylnaphthalenesulphonate salts, such as sodium dibutylnaphthalenesulphonate; dialkyl esters of sulphosuccinate salts, such as sodium di(2-ethylhexyl) sulphosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants, sticking agents, and the like.

Each of the above formulations can be prepared as a package containing the fungicides together with other ingredients of the formulation (diluents, emulsifiers, surfactants, etc.). The formulations can also be prepared by a tank mix method, in which the ingredients are obtained separately and combined at the grower site.

These formulations can be applied to the areas where control is desired by conventional methods. Dust and liquid compositions, for example, can be applied by the use of power-dusters, broom and hand sprayers and spray dusters. The formulations can also be applied from airplanes as a dust or a spray or by rope wick applications. Both solid and liquid formulations may also be applied to the soil in the locus of the plant to be treated allowing the active ingredient to penetrate the plant through the roots. The formulations of the invention may also be used for dressing applications on plant propagation material to provide protection against fungus infections on the plant propagation material as well as against phytopathogenic fungi occurring in the soil. Suitably, the active ingredient may be applied to plant propagation material to be protected by impregnating the plant propagation material, in particular, seeds, either with a liquid formulation of the fungicide or coating it with a solid formulation. In special cases, other types of application are also possible, for example, the specific treatment of plant cuttings or twigs serving propagation.

Suitably, the agrochemical compositions and formulations of the present invention are applied prior to disease development. Rates and frequency of use of the formulations are those conventionally used in the art and will depend on the risk of infestation by the fungal pathogen.

The compositions and formulations of the present invention can also be used in combination with other active ingredients (compound II), e.g. other fungicides, and/or insecticides, and/or acaricides, and/or nematocides, and/or molluscicides, and/or biologicals, and/or plant growth regulators. Such mixtures, and the use of such mixtures to control weeds and/or undesired plant growth form yet further aspects of the invention.

The following mixtures of the compound of formula I with active ingredients (compound II) are preferred:
an adjuvant selected from the group of substances consisting of petroleum oils (alternative name) (628);
an insect control active substance selected from abamectin, acequinocyl, acetamiprid, acetoprole, acrinathrin, acynonapyr, afidopyropen, afoxolaner, alanycarb, allethrin, alpha-cypermethrin, alphamethrin, amidoflumet, aminocarb, azocyclotin, bensultap, benzoximate, benzpyrimoxan, betacyfluthrin, beta-cypermethrin, bifenazate, bifenthrin, binapacryl, bioallethrin, S-bioallethrin, bioresmethrin, bistrifluron, broflanilide, brofluthrinate, bromophos-ethyl, buprofezine, butocarboxim, cadusafos, carbaryl, carbosulfan, cartap, CAS number: 1632218-00-8, CAS number: 1808115-49-2, CAS number: 2032403-97-5, CAS number: 2044701-44-0, CAS number: 2128706-05-6, CAS number: 2095470-94-1, CAS number: 2377084-09-6, CAS number: 1445683-71-5, CAS number: 2408220-94-8, CAS number: 2408220-91-5, CAS number: 1365070-72-9, CAS number: 2171099-09-3, CAS number: 2396747-83-2, CAS number: 2133042-31-4, CAS number: 2133042-44-9, CAS number: 1445684-82-1, CAS number: 1922957-45-6, CAS number: 1922957-46-7, CAS number: 1922957-47-8, CAS number: 1922957-48-9, CAS number: 2415706-16-8, CAS number: 1594624-87-9, CAS number: 1594637-65-6, CAS number: 1594626-19-3, CAS number: 1990457-52-7, CAS number: 1990457-55-0, CAS number: 1990457-57-2, CAS number: 1990457-77-6, CAS number: 1990457-66-3, CAS number: 1990457-85-6, CAS number: 2220132-55-6, CAS number: 1255091-74-7, CAS number: RNA (Leptinotarsa decemlineata-specific recombinant double-stranded interfering GS2), CAS number: 2719848-60-7, CAS number: 1956329-03-5, chlorantraniliprole, chlordane, chlorfenapyr, chloroprallethrin, chromafenozide, clenpirin, cloethocarb, clothianidin, 2-chlorophenyl N-methylcarbamate (CPMC), cyanofenphos, cyantraniliprole, cyclaniliprole, cyclobutrifluram, cycloprothrin, cycloxaprid, cyenopyrafen, cyetpyrafen (or etpyrafen), cyflumetofen, cyfluthrin, cyhalodiamide, cyhalothrin, cypermethrin, cyphenothrin, cyproflanilide, cyromazine, deltamethrin, diafenthiuron, dialifos, dibrom, dicloromezotiaz, diflovidazine, diflubenzuron, dimpropyridaz, dinactin, dinocap, dinotefuran, dioxabenzofos, emamectin (or emamectin benzoate), empenthrin, epsilon-momfluorothrin, epsilon-metofluthrin, esfenvalerate, ethion, ethiprole, etofenprox, etoxazole, famphur, fenazaquin, fenfluthrin, fenitrothion, fenmezoditiaz, fenobucarb, fenothiocarb, fenoxycarb, fenpropathrin, fenpyroximate, fensulfothion, fenthion, fentinacetate, fenvalerate, fipronil, flometoquin, flonicamid, fluacrypyrim, fluazaindolizine, fluazuron, flubendiamide, flubenzimine, fluchlordiniliprole, flucitrinate, flucycloxuron, flucythrinate, fluensulfone, flufenerim, flufenprox, flufiprole, fluhexafon, flumethrin, fluopyram, flupentiofenox flupyradifurone, flupyroxystrobin, flupyrimin, fluralaner, fluvalinate, fluxametamide, fosthiazate, gamma-cyhalothrin, guadipyr, halofenozide, halfenprox, heptafluthrin, hexythiazox, hydramethylnon, imicyafos, imidacloprid, imiprothrin, Indazapyroxamet, indoxacarb, iodomethane, iprodione, isocycloseram, isothioate, ivermectin, kappa-bifenthrin, kappa-tefluthrin, lambda-cyhalothrin, ledprona, lepimectin, lotilaner, lufenuron, metaflumizone, metaldehyde, metam, methomyl, methoxyfenozide, metofluthrin, metolcarb, mexacarbate, milbemectin, momfluorothrin, niclosamide, nicofluprole, nitenpyram, nithiazine, omethoate, oxamyl, oxazosulfyl, parathion-ethyl, permethrin, phenothrin, phosphocarb, piperonylbutoxide, pirimicarb, pirimiphos-ethyl, pirimiphos-methyl, Polyhedrosis virus, prallethrin, profenofos, profluthrin, propargite, propetamphos, propoxur, prothiophos, protrifenbute, pyflubumide, pymetrozine, pyraclofos, pyrafluprole, pyridaben, pyridalyl, pyrifluquinazon, pyrimidifen, pyriminostrobin, pyriprole, pyriproxyfen, resmethrin, sarolaner, selamectin, silafluofen, spinetoram, spinosad, spirobudifen, spirodiclofen, spiromesifen, spiropidion, spirotetramat, spidoxamat, sulfoxaflor, tebufenozide, tebufenpyrad, tebupirimiphos, tefluthrin, temephos, tetrachlorantraniliprole, tetradiphon, tetramethrin, tetramethylfluthrin, tetranactin, tetraniliprole, theta-cypermethrin, thiacloprid, thiamethoxam, thiocyclam, thiodicarb, thiofanox, thiometon, thiosultap, tigolaner, tiorantraniliprole, tioxazafen, tolfenpyrad, toxaphene, tralomethrin, transfluthrin, triazamate, triazophos, trichlorfon, trichloronate, trichlorphon, trifluenfuronate, triflumezopyrim, tyclopyrazoflor, zeta-Cypermethrin, Extract of seaweed and fermentation product derived from melasse, Extract of seaweed and fermentation product derived from melasse comprising urea, amino acids, potassium and molybdenum and EDTA-chelated manganese, Extract of seaweed and fermented plant products, Extract of seaweed and fermented plant products comprising phytohormones, vitamins, EDTA-chelated copper, zinc, and iron, azadirachtin, Bacillus aizawai, Bacillus chitinosporus AQ746 (NRRL Accession No B-21 618), Bacillus firmus, Bacillus kurstaki, Bacillus mycoides AQ726 (NRRL Accession No. B-21664), Bacillus pumilus (NRRL Accession No B-30087), Bacillus pumilus AQ717 (NRRL Accession No. B-21662), Bacillus sp. AQ178 (ATCC Accession No. 53522), Bacillus sp. AQ175 (ATCC Accession No. 55608), Bacillus sp. AQ177 (ATCC Accession No. 55609), Bacillus subtilis unspecified, Bacillus subtilis AQ153 (ATCC Accession No. 55614), Bacillus subtilis AQ30002 (NRRL Accession No. B-50421), Bacillus subtilis AQ30004 (NRRL Accession No. B-50455), Bacillus subtilis AQ713 (NRRL Accession No. B-21661), Bacillus subtilis AQ743 (NRRL Accession No. B-21665), Bacillus thuringiensis AQ52 (NRRL Accession No. B-21619), Bacillus thuringiensis BD #32 (NRRL Accession No B-21530), Bacillus thuringiensis subspec. kurstaki BMP 123, Beauveria bassiana, D-limonene, Granulovirus, Harpin, Helicoverpa armigera Nucleopolyhedrovirus, Helicoverpa zea Nucleopolyhedrovirus, Heliothis virescens Nucleopolyhedrovirus, Heliothis punctigera Nucleopolyhedrovirus, Metarhizium spp., Muscodor albus 620 (NRRL Accession No. 30547), Muscodor roseus A3-5 (NRRL Accession No. 30548), Neem tree based products, Paecilomyces fumosoroseus, Paecilomyces lilacinus, Pasteuria nishizawae, Pasteuria penetrans, Pasteuria ramosa, Pasteuria thornei, Pasteuria usgae, P-cymene, Plutella xylostella Granulosis virus, Plutella xylostella Nucleopolyhedrovirus, Polyhedrosis virus, pyrethrum, QRD 420 (a terpenoid blend), QRD 452 (a terpenoid blend), QRD 460 (a terpenoid blend), Quillaja saponaria, Rhodococcus globerulus AQ719 (NRRL Accession No B-21663), Spodoptera frugiperda Nucleopolyhedrovirus, Streptomyces galbus (NRRL Accession No. 30232), Streptomyces sp. (NRRL Accession No. B-30145), terpenoid blend, and Verticillium spp.;
an algicide selected from the group of substances consisting of bethoxazin [CCN], copper dioctanoate (IUPAC name) (170), copper sulfate (172), cybutryne [CCN], dichlone (1052), dichlorophen (232), endothal (295), fentin (347), hydrated lime [CCN], nabam (566), quinoclamine (714), quinonamid (1379), simazine (730), triphenyltin acetate (IUPAC name) (347) and triphenyltin hydroxide (IUPAC name) (347);
an anthelmintic selected from the group of substances consisting of abamectin (1), crufomate (1011), doramectin (alternative name) [CCN], emamectin (291), emamectin benzoate (291), eprinomectin (alternative name) [CCN], ivermectin (alternative name) [CCN], milbemycin oxime (alternative name) [CCN], moxidectin (alternative name) [CCN], piperazine [CCN], selamectin (alternative name) [CCN], spinosad (737) and thiophanate (1435);
an avicide selected from the group of substances consisting of chloralose (127), endrin (1122), fenthion (346), pyridin-4-amine (IUPAC name) (23) and strychnine (745);
a bactericide selected from the group of substances consisting of 1-hydroxy-1H-pyridine-2-thione (IUPAC name) (1222), 4-(quinoxalin-2-ylamino)benzenesulfonamide (IUPAC name) (748), 8-hydroxyquinoline sulfate (446), bronopol (97), copper dioctanoate (IUPAC name) (170), copper hydroxide (IUPAC name) (169), cresol [CCN], dichlorophen (232), dipyrithione (1105), dodicin (1112), fenaminosulf (1144), formaldehyde (404), hydrargaphen (alternative name) [CCN], kasugamycin (483), kasugamycin hydrochloride hydrate (483), nickel bis(dimethyldithiocarbamate) (IUPAC name) (1308), nitrapyrin (580), octhilinone (590), oxolinic acid (606), oxytetracycline (611), potassium hydroxyquinoline sulfate (446), probenazole (658), streptomycin (744), streptomycin sesquisulfate (744), tecloftalam (766), and thiomersal (alternative name) [CCN];
a biological agent selected from the group of substances consisting of Adoxophyes orana GV (alternative name) (12), Agrobacterium radiobacter (alternative name) (13), Amblyseius spp. (alternative name) (19), Anagrapha falcifera NPV (alternative name) (28), Anagrus atomus (alternative name) (29), Aphelinus abdominalis (alternative name) (33), Aphidius colemani (alternative name) (34), Aphidoletes aphidimyza (alternative name) (35), Autographa californica NPV (alternative name) (38), Bacillus firmus (alternative name) (48), Bacillus sphaericus Neide (scientific name) (49), Bacillus thuringiensis Berliner (scientific name) (51), Bacillus thuringiensis subsp. aizawai (scientific name) (51), Bacillus thuringiensis subsp. israelensis (scientific name) (51), Bacillus thuringiensis subsp. japonensis (scientific name) (51), Bacillus thuringiensis subsp. kurstaki (scientific name) (51), Bacillus thuringiensis subsp. tenebrionis (scientific name) (51), Beauveria bassiana (alternative name) (53), Beauveria brongniartii (alternative name) (54), Chrysoperla carnea (alternative name) (151), Cryptolaemus montrouzieri (alternative name) (178), Cydia pomonella GV (alternative name) (191), Dacnusa sibirica (alternative name) (212), Diglyphus isaea (alternative name) (254), Encarsia formosa (scientific name) (293), Eretmocerus eremicus (alternative name) (300), Helicoverpa zea NPV (alternative name) (431), Heterorhabditis bacteriophora and H. megidis (alternative name) (433), Hippodamia convergens (alternative name) (442), Leptomastix dactylopii (alternative name) (488), Macrolophus caliginosus (alternative name) (491), Mamestra brassicae NPV (alternative name) (494), Metaphycus helvolus (alternative name) (522), Metarhizium anisopliae var. acridum (scientific name) (523), Metarhizium anisopliae var. anisopliae (scientific name) (523), Neodiprion sertifer NPV and N. lecontei NPV (alternative name) (575), Orius spp. (alternative name) (596), Paecilomyces fumosoroseus (alternative name) (613), Phytoseiulus persimilis (alternative name) (644), Spodoptera exigua multicapsid nuclear polyhedrosis virus (scientific name) (741), Steinernema bibionis (alternative name) (742), Steinernema carpocapsae (alternative name) (742), Steinernema feltiae (alternative name) (742), Steinernema glaseri (alternative name) (742), Steinernema riobrave (alternative name) (742), Steinernema riobravis (alternative name) (742), Steinernema scapterisci (alternative name) (742), Steinernema spp. (alternative name) (742), Trichogramma spp. (alternative name) (826), Typhlodromus occidentalis (alternative name) (844) and Verticillium lecanii (alternative name) (848);
a soil sterilant selected from the group of substances consisting of iodomethane (IUPAC name) (542) and methyl bromide (537);
a chemosterilant selected from the group of substances consisting of apholate [CCN], bisazir (alternative name) [CCN], busulfan (alternative name) [CCN], diflubenzuron (250), dimatif (alternative name) [CCN], hemel [CCN], hempa [CCN], metepa [CCN], methiotepa [CCN], methyl apholate [CCN], morzid [CCN], penfluron (alternative name) [CCN], tepa [CCN], thiohempa (alternative name) [CCN], thiotepa (alternative name) [CCN], tretamine (alternative name) [CCN] and uredepa (alternative name) [CCN];
an insect pheromone selected from the group of substances consisting of (E)-dec-5-en-1-yl acetate with (E)-dec-5-en-1-ol (IUPAC name) (222), (E)-tridec-4-en-1-yl acetate (IUPAC name) (829), (E)-6-methylhept-2-en-4-ol (IUPAC name) (541), (E,Z)-tetradeca-4,10-dien-1-yl acetate (IUPAC name) (779), (Z)-dodec-7-en-1-yl acetate (IUPAC name) (285), (Z)-hexadec-11-enal (IUPAC name) (436), (Z)-hexadec-11-en-1-yl acetate (IUPAC name) (437), (Z)-hexadec-13-en-11-yn-1-yl acetate (IUPAC name) (438), (Z)-icos-13-en-10-one (IUPAC name) (448), (Z)-tetradec-7-en-1-al (IUPAC name) (782), (Z)-tetradec-9-en-1-ol (IUPAC name) (783), (Z)-tetradec-9-en-1-yl acetate (IUPAC name) (784), (7E,9Z)-dodeca-7,9-dien-1-yl acetate (IUPAC name) (283), (9Z,11E)-tetradeca-9,11-dien-1-yl acetate (IUPAC name) (780), (9Z,12E)-tetradeca-9,12-dien-1-yl acetate (IUPAC name) (781), 14-methyloctadec-1-ene (IUPAC name) (545), 4-methylnonan-5-ol with 4-methylnonan-5-one (IUPAC name) (544), alpha-multistriatin (alternative name) [CCN], brevicomin (alternative name) [CCN], codlelure (alternative name) [CCN], codlemone (alternative name) (167), cuelure (alternative name) (179), disparlure (277), dodec-8-en-1-yl acetate (IUPAC name) (286), dodec-9-en-1-yl acetate (IUPAC name) (287), dodeca-8, 10-dien-1-yl acetate (IUPAC name) (284), dominicalure (alternative name) [CCN], ethyl 4-methyloctanoate (IUPAC name) (317), eugenol (alternative name) [CCN], frontalin (alternative name) [CCN], Gossyplure® (alternative name; 1:1 mixture of the (Z,E) and (Z,Z) isomers of hexadeca-7,11-dien-1-yl-acetate) (420), grandlure (421), grandlure I (alternative name) (421), grandlure II (alternative name) (421), grandlure III (alternative name) (421), grandlure IV (alternative name) (421), hexalure [CCN], ipsdienol (alternative name) [CCN], ipsenol (alternative name) [CCN], japonilure (alternative name) (481), lineatin (alternative name) [CCN], litlure (alternative name) [CCN], looplure (alternative name) [CCN], medlure [CCN], megatomoic acid (alternative name) [CCN], methyl eugenol (alternative name) (540), muscalure (563), octadeca-2,13-dien-1-yl acetate (IUPAC name) (588), octadeca-3,13-dien-1-yl acetate (IUPAC name) (589), orfralure (alternative name) [CCN], oryctalure (alternative name) (317), ostramone (alternative name) [CCN], siglure [CCN], sordidin (alternative name) (736), sulcatol (alternative name) [CCN], tetradec-11-en-1-yl acetate (IUPAC name) (785), trimedlure (839), trimedlure A (alternative name) (839), trimedlure B1 (alternative name) (839), trimedlure B2 (alternative name) (839), trimedlure C (alternative name) (839) and trunc-call (alternative name) [CCN];
an insect repellent selected from the group of substances consisting of 2-(octylthio)ethanol (IUPAC name) (591), butopyronoxyl (933), butoxy (polypropylene glycol) (936), dibutyl adipate (IUPAC name) (1046), dibutyl phthalate (1047), dibutyl succinate (IUPAC name) (1048), diethyltoluamide [CCN], dimethyl carbate [CCN], dimethyl phthalate [CCN], ethyl hexanediol (1137), hexamide [CCN], methoquin-butyl (1276), methylneodecanamide [CCN], oxamate [CCN] and picaridin [CCN];
a molluscicide selected from the group of substances consisting of bis(tributyltin) oxide (IUPAC name) (913), bromoacetamide [CCN], calcium arsenate [CCN], cloethocarb (999), copper acetoarsenite [CCN], copper sulfate (172), fentin (347), ferric phosphate (IUPAC name) (352), metaldehyde (518), methiocarb (530), niclosamide (576), niclosamide-olamine (576), pentachlorophenol (623), sodium pentachlorophenoxide (623), tazimcarb (1412), thiodicarb (799), tributyltin oxide (913), trifenmorph (1454), trimethacarb (840), triphenyltin acetate (IUPAC name) (347), triphenyltin hydroxide (IUPAC name) (347), and pyriprole [394730-71-3];
a nematicide selected from the group of substances consisting of AKD-3088 (compound code), 1,2-dibromo-3-chloropropane (IUPAC/Chemical Abstracts name) (1045), 1,2-dichloropropane (IUPAC/Chemical Abstracts name) (1062), 1,2-dichloropropane with 1,3-dichloropropene (IUPAC name) (1063), 1,3-dichloropropene (233), 3,4-dichlorotetrahydrothiophene 1,1-dioxide (IUPAC/Chemical Abstracts name) (1065), 3-(4-chlorophenyl)-5-methylrhodanine (IUPAC name) (980), 5-methyl-6-thioxo-1,3,5-thiadiazinan-3-ylacetic acid (IUPAC name) (1286), 6-isopentenylaminopurine (alternative name) (210), abamectin (1), acetoprole [CCN], alanycarb (15), aldicarb (16), aldoxycarb (863), AZ 60541 (compound code), benclothiaz [CCN], benomyl (62), butylpyridaben (alternative name), cadusafos (109), carbofuran (118), carbon disulfide (945), carbosulfan (119), chloropicrin (141), chlorpyrifos (145), cloethocarb (999), cytokinins (alternative name) (210), dazomet (216), DBCP (1045), DCIP (218), diamidafos (1044), dichlofenthion (1051), dicliphos (alternative name), dimethoate (262), doramectin (alternative name) [CCN], emamectin (291), emamectin benzoate (291), eprinomectin (alternative name) [CCN], ethoprophos (312), ethylene dibromide (316), fenamiphos (326), fenpyrad (alternative name), fensulfothion (1158), fosthiazate (408), fosthietan (1196), furfural (alternative name) [CCN], GY-81 (development code) (423), heterophos [CCN], iodomethane (IUPAC name) (542), isamidofos (1230), isazofos (1231), ivermectin (alternative name) [CCN], kinetin (alternative name) (210), mecarphon (1258), metam (519), metam-potassium (alternative name) (519), metam-sodium (519), methyl bromide (537), methyl isothiocyanate (543), milbemycin oxime (alternative name) [CCN], moxidectin (alternative name) [CCN], Myrothecium verrucaria composition (alternative name) (565), NC-184 (compound code), oxamyl (602), phorate (636), phosphamidon (639), phosphocarb [CCN], sebufos (alternative name), selamectin (alternative name) [CCN], spinosad (737), terbam (alternative name), terbufos (773), tetrachlorothiophene (IUPAC/Chemical Abstracts name) (1422), thiafenox (alternative name), thionazin (1434), triazophos (820), triazuron (alternative name), xylenols [CCN], YI-5302 (compound code), zeatin (alternative name) (210), fluensulfone [318290-98-1], and fluopyram;
a nitrification inhibitor selected from the group of substances consisting of potassium ethylxanthate [CCN] and nitrapyrin (580);
a plant activator selected from the group of substances consisting of acibenzolar (6), acibenzolar-S-methyl (6), probenazole (658) and Reynoutria sachalinensis extract (alternative name) (720);
a rodenticide selected from the group of substances consisting of 2-isovalerylindan-1,3-dione (IUPAC name) (1246), 4-(quinoxalin-2-ylamino)benzenesulfonamide (IUPAC name) (748), alpha-chlorohydrin [CCN], aluminium phosphide (640), antu (880), arsenous oxide (882), barium carbonate (891), bisthiosemi (912), brodifacoum (89), bromadiolone (or alpha-bromadiolone), bromethalin (92), calcium cyanide (444), chloralose (127), chlorophacinone (140), cholecalciferol (alternative name) (850), coumachlor (1004), coumafuryl (1005), coumatetralyl (175), crimidine (1009), difenacoum (246), difethialone (249), diphacinone (273), ergocalciferol (301), flocoumafen (357), fluoroacetamide (379), flupropadine (1183), flupropadine hydrochloride (1183), gamma-HCH (430), HCH (430), hydrogen cyanide (444), iodomethane (IUPAC name) (542), lindane (430), magnesium phosphide (IUPAC name) (640), methyl bromide (537), norbormide (1318), phosacetim (1336), phosphine (IUPAC name) (640), phosphorus [CCN], pindone (1341), potassium arsenite [CCN], pyrinuron (1371), scilliroside (1390), sodium arsenite [CCN], sodium cyanide (444), sodium fluoroacetate (735), strychnine (745), thallium sulfate [CCN], warfarin (851) and zinc phosphide (640);
a synergist selected from the group of substances consisting of 2-(2-butoxyethoxy)ethyl piperonylate (IUPAC name) (934), 5-(1,3-benzodioxol-5-yl)-3-hexylcyclohex-2-enone (IUPAC name) (903), farnesol with nerolidol (alternative name) (324), MB-599 (development code) (498), MGK 264 (development code) (296), piperonyl butoxide (649), piprotal (1343), propyl isomer (1358), S421 (development code) (724), sesamex (1393), sesasmolin (1394) and sulfoxide (1406),
an animal repellent selected from the group of substances consisting of anthraquinone (32), chloralose (127), copper naphthenate [CCN], copper oxychloride (171), diazinon (227), dicyclopentadiene (chemical name) (1069), guazatine (422), guazatine acetates (422), methiocarb (530), pyridin-4-amine (IUPAC name) (23), thiram (804), trimethacarb (840), zinc naphthenate [CCN] and ziram (856);
a virucide selected from the group of substances consisting of imanin (alternative name) [CCN] and ribavirin (alternative name) [CCN];
a wound protectant selected from the group of substances consisting of mercuric oxide (512), octhilinone (590) and thiophanate-methyl (802);
a biologically active substance selected from 1,1-bis(4-chlorophenyl)-2-ethoxyethanol, 2,4-dichlorophenyl benzenesulfonate, 2-fluoro-N-methyl-N-1-naphthylacetamide, 4-chlorophenyl phenyl sulfone, acetoprole, aldoxycarb, amidithion, amidothioate, amiton, amiton hydrogen oxalate, amitraz, aramite, arsenous oxide, azobenzene, azothoate, benomyl, benoxafos, benzyl benzoate, bixafen, brofenvalerate, bromocyclen, bromophos, bromopropylate, buprofezin, butocarboxim, butoxycarboxim, butylpyridaben, calcium polysulfide, camphechlor, carbanolate, carbophenothion, cymiazole, chinomethionat, chlorbenside, chlordimeform, chlordimeform hydrochloride, chlorfenethol, chlorfenson, chlorfensulfide, chlorobenzilate, chloromebuform, chloromethiuron, chloropropylate, chlorthiophos, cinerin I, cinerin II, cinerins, closantel, coumaphos, crotamiton, crotoxyphos, cufraneb, cyanthoate, DCPM, DDT, demephion, demephion-O, demephion-S, demeton-methyl, demeton-O, demeton-O-methyl, demeton-S, demeton-S-methyl, demeton-S-methylsulfon, dichlofluanid, dichlorvos, dicliphos, dienochlor, dimefox, dinex, dinex-diclexine, dinocap-4, dinocap-6, dinocton, dinopenton, dinosulfon, dinoterbon, dioxathion, diphenyl sulfone, disulfiram, DNOC, dofenapyn, doramectin, endothion, eprinomectin, ethoate-methyl, etrimfos, fenazaflor, fenbutatin oxide, fenothiocarb, fenpyrad, fenpyroximate, fenpyrazamine, fenson, fentrifanil, flubenzimine, flucycloxuron, fluenetil, fluorbenside, FMC 1137, formetanate, formetanate hydrochloride, formparanate, gamma-HCH, glyodin, halfenprox, hexadecyl cyclopropanecarboxylate, isocarbophos, jasmolin I, jasmolin II, jodfenphos, lindane, malonoben, mecarbam, mephosfolan, mesulfen, methacrifos, methyl bromide, metolcarb, mexacarbate, milbemycin oxime, mipafox, monocrotophos, morphothion, moxidectin, naled, 4-chloro-2-(2-chloro-2-methyl-propyl)-5-[(6-iodo-3-pyridyl) methoxy]pyridazin-3-one, nifluridide, nikkomycins, nitrilacarb, nitrilacarb 1:1 zinc chloride complex, omethoate, oxydeprofos, oxydisulfoton, pp′-DDT, parathion, permethrin, phenkapton, phosalone, phosfolan, phosphamidon, polychloroterpenes, polynactins, proclonol, promacyl, propoxur, prothidathion, prothoate, pyrethrin I, pyrethrin II, pyrethrins, pyridaphenthion, pyrimitate, quinalphos, quintiofos, R-1492, phosglycin, rotenone, schradan, sebufos, selamectin, sophamide, SSI-121, sulfiram, sulfluramid, sulfotep, sulfur, diflovidazin, tau-fluvalinate, TEPP, terbam, tetradifon, tetrasul, thiafenox, thiocarboxime, thiofanox, thiometon, thioquinox, thuringiensin, triamiphos, triarathene, triazophos, triazuron, trifenofos, trinactin, vamidothion, vaniliprole, bethoxazin, copper dioctanoate, copper sulfate, cybutryne, dichlone, dichlorophen, endothal, fentin, hydrated lime, nabam, quinoclamine, quinonamid, simazine, triphenyltin acetate, triphenyltin hydroxide, crufomate, piperazine, thiophanate, chloralose, fenthion, pyridin-4-amine, strychnine, 1-hydroxy-1H-pyridine-2-thione, 4-(quinoxalin-2-ylamino)benzenesulfonamide, 8-hydroxyquinoline sulfate, bronopol, copper hydroxide, cresol, dipyrithione, dodicin, fenaminosulf, formaldehyde, hydrargaphen, kasugamycin, kasugamycin hydrochloride hydrate, nickel bis(dimethyldithiocarbamate), nitrapyrin, octhilinone, oxolinic acid, oxytetracycline, potassium hydroxyquinoline sulfate, probenazole, streptomycin, streptomycin sesquisulfate, tecloftalam, thiomersal, Adoxophyes orana GV, Agrobacterium radiobacter, Amblyseius spp., Anagrapha falcifera NPV, Anagrus atomus, Aphelinus abdominalis, Aphidius colemani, Aphidoletes aphidimyza, Autographa californica NPV, Bacillus sphaericus Neide, Beauveria brongniartii, Chrysoperla carnea, Cryptolaemus montrouzieri, Cydia pomonella GV, Dacnusa sibirica, Diglyphus isaea, Encarsia formosa, Eretmocerus eremicus, Heterorhabditis bacteriophora and H. megidis, Hippodamia convergens, Leptomastix dactylopii, Macrolophus caliginosus, Mamestra brassicae NPV, Metaphycus helvolus, Metarhizium anisopliae var. acridum, Metarhizium anisopliae var. anisopliae, Neodiprion sertifer NPV and N. lecontei NPV, Orius spp., Paecilomyces fumosoroseus, Phytoseiulus persimilis, Steinernema bibionis, Steinernema carpocapsae, Steinernema feltiae, Steinernema glaseri, Steinernema riobrave, Steinernema riobravis, Steinernema scapterisci, Steinernema spp., Trichogramma spp., Typhlodromus occidentalis, Verticillium lecanii, apholate, bisazir, busulfan, dimatif, hemel, hempa, metepa, methiotepa, methyl apholate, morzid, penfluron, tepa, thiohempa, thiotepa, tretamine, uredepa, (E)-dec-5-en-1-yl acetate with (E)-dec-5-en-1-ol, (E)-tridec-4-en-1-yl acetate, (E)-6-methylhept-2-en-4-ol, (E,Z)-tetradeca-4,10-dien-1-yl acetate, (Z)-dodec-7-en-1-yl acetate, (Z)-hexadec-11-enal, (Z)-hexadec-11-en-1-yl acetate, (Z)-hexadec-13-en-11-yn-1-yl acetate, (Z)-icos-13-en-10-one, (Z)-tetradec-7-en-1-al, (Z)-tetradec-9-en-1-ol, (Z)-tetradec-9-en-1-yl acetate, (7E,9Z)-dodeca-7,9-dien-1-yl acetate, (9Z,11E)-tetradeca-9,11-dien-1-yl acetate, (9Z,12E)-tetradeca-9,12-dien-1-yl acetate, 14-methyloctadec-1-ene, 4-methylnonan-5-ol with 4-methylnonan-5-one, alpha-multistriatin, brevicomin, codlelure, codlemone, cuelure, disparlure, dodec-8-en-1-yl acetate, dodec-9-en-1-yl acetate, dodeca-8, 10-dien-1-yl acetate, dominicalure, ethyl 4-methyloctanoate, eugenol, frontalin, grandlure, grandlure I, grandlure II, grandlure III, grandlure IV, hexalure, ipsdienol, ipsenol, japonilure, lineatin, litlure, looplure, medlure, megatomoic acid, methyl eugenol, muscalure, octadeca-2,13-dien-1-yl acetate, octadeca-3,13-dien-1-yl acetate, orfralure, oryctalure, ostramone, siglure, sordidin, sulcatol, tetradec-11-en-1-yl acetate, trimedlure, trimedlure A, trimedlure B1, trimedlure B2, trimedlure C, trunc-call, 2-(octylthio)ethanol, butopyronoxyl, butoxy (polypropylene glycol), dibutyl adipate, dibutyl phthalate, dibutyl succinate, diethyltoluamide, dimethyl carbate, dimethyl phthalate, ethyl hexanediol, hexamide, methoquin-butyl, methylneodecanamide, oxamate, picaridin, 1-dichloro-1-nitroethane, 1,1-dichloro-2,2-bis(4-ethylphenyl)ethane, 1,2-dichloropropane with 1,3-dichloropropene, 1-bromo-2-chloroethane, 2,2,2-trichloro-1-(3,4-dichlorophenyl)ethyl acetate, 2,2-dichlorovinyl 2-ethylsulfinylethyl methyl phosphate, 2-(1,3-dithiolan-2-yl)phenyl dimethylcarbamate, 2-(2-butoxyethoxy)ethyl thiocyanate, 2-(4,5-dimethyl-1,3-dioxolan-2-yl)phenyl methylcarbamate, 2-(4-chloro-3,5-xylyloxy)ethanol, 2-chlorovinyl diethyl phosphate, 2-imidazolidone, 2-isovalerylindan-1,3-dione, 2-methyl(prop-2-ynyl)aminophenyl methylcarbamate, 2-thiocyanatoethyl laurate, 3-bromo-1-chloroprop-1-ene, 3-methyl-1-dimethylcarbamate, phenylpyrazol-5-yl 4-methyl(prop-2-ynyl)amino-3,5-xylyl methylcarbamate, 5,5-dimethyl-3-oxocyclohex-1-enyl dimethylcarbamate, acethion, acrylonitrile, aldrin, allosamidin, allyxycarb, alpha-ecdysone, aluminium phosphide, aminocarb, anabasine, athidathion, azamethiphos, Bacillus thuringiensis delta endotoxins, barium hexafluorosilicate, barium polysulfide, barthrin, Bayer 22/190, Bayer 22408, beta-cyfluthrin, beta-cypermethrin, bioethanomethrin, biopermethrin, bis(2-chloroethyl)ether, borax, bromfenvinfos, bromo-DDT, bufencarb, butacarb, butathiofos, butonate, calcium arsenate, calcium cyanide, carbon disulfide, carbon tetrachloride, cartap hydrochloride, cevadine, chlorbicyclen, chlordane, chlordecone, chloroform, chloropicrin, chlorphoxim, chlorprazophos, cis-resmethrin, cismethrin, clocythrin, copper acetoarsenite, copper arsenate, copper oleate, coumithoate, cryolite, CS 708, cyanofenphos, cyanophos, cyclethrin, cythioate, d-tetramethrin, DAEP, dazomet, decarbofuran, diamidafos, dicapthon, dichlofenthion, dicresyl, dicyclanil, dieldrin, diethyl 5-methylpyrazol-3-yl phosphate, dilor, dimefluthrin, dimetan, dimethrin, dimethylvinphos, dimetilan, dinoprop, dinosam, dinoseb, diofenolan, dioxabenzofos, dithicrofos, DSP, ecdysterone, EI 1642, EMPC, EPBP, etaphos, ethiofencarb, ethyl formate, ethylene dibromide, ethylene dichloride, ethylene oxide, EXD, fenchlorphos, fenethacarb, fenitrothion, fenoxacrim, fenpirithrin, fensulfothion, fenthion-ethyl, flucofuron, fosmethilan, fospirate, fosthietan, furathiocarb, furethrin, guazatine, guazatine acetates, sodium tetrathiocarbonate, halfenprox, HCH, HEOD, heptachlor, heterophos, HHDN, hydrogen cyanide, hyquincarb, IPSP, isazofos, isobenzan, isodrin, isofenphos, isolane, isoprothiolane, isoxathion, juvenile hormone I, juvenile hormone II, juvenile hormone III, kelevan, kinoprene, lead arsenate, leptophos, lirimfos, lythidathion, m-cumenyl methylcarbamate, magnesium phosphide, mazidox, mecarphon, menazon, mercurous chloride, mesulfenfos, metam, metam-potassium, metam-sodium, methanesulfonyl fluoride, methocrotophos, methoprene, methothrin, methoxychlor, methyl isothiocyanate, methylchloroform, methylene chloride, metoxadiazone, mirex, naftalofos, naphthalene, NC-170, nicotine, nicotine sulfate, nithiazine, nornicotine, O-5-dichloro-4-iodophenyl O-ethyl ethylphosphonothioate, O,O-diethyl O-4-methyl-2-oxo-2H-chromen-7-yl phosphorothioate, O,O-diethyl O-6-methyl-2-propylpyrimidin-4-yl phosphorothioate, O,O,O′,O′-tetrapropyl dithiopyrophosphate, oleic acid, para-dichlorobenzene, parathion-methyl, pentachlorophenol, pentachlorophenyl laurate, PH 60-38, phenkapton, phosnichlor, phosphine, phoxim-methyl, pirimetaphos, polychlorodicyclopentadiene isomers, potassium arsenite, potassium thiocyanate, precocene I, precocene II, precocene III, primidophos, profluthrin, promecarb, prothiofos, pyrazophos, pyresmethrin, quassia, quinalphos-methyl, quinothion, rafoxanide, resmethrin, rotenone, kadethrin, ryania, ryanodine, sabadilla, schradan, sebufos, SI-0009, thiapronil, sodium arsenite, sodium cyanide, sodium fluoride, sodium hexafluorosilicate, sodium pentachlorophenoxide, sodium selenate, sodium thiocyanate, sulcofuron, sulcofuron-sodium, sulfuryl fluoride, sulprofos, tar oils, tazimcarb, TDE, tebupirimfos, temephos, terallethrin, tetrachloroethane, thicrofos, thiocyclam, thiocyclam hydrogen oxalate, thionazin, thiosultap, thiosultap-sodium, tralomethrin, transpermethrin, triazamate, trichlormetaphos-3, trichloronat, trimethacarb, tolprocarb, triclopyricarb, triprene, veratridine, veratrine, XMC, zetamethrin, zinc phosphide, zolaprofos, meperfluthrin, tetramethylfluthrin, bis(tributyltin) oxide, bromoacetamide, ferric phosphate, niclosamide-olamine, tributyltin oxide, pyrimorph, trifenmorph, 1,2-dibromo-3-chloropropane, 1,3-dichloropropene, 3,4-dichlorotetrahydrothiophene 1,1-dioxide, 3-(4-chlorophenyl)-5-methylrhodanine, 5-methyl-6-thioxo-1,3,5-thiadiazinan-3-ylacetic acid, 6-isopentenylaminopurine, anisiflupurin, benclothiaz, cytokinins, DCIP, furfural, isamidofos, kinetin, Myrothecium verrucaria composition, tetrachlorothiophene, xylenols, zeatin, potassium ethylxanthate, acibenzolar, acibenzolar-S-methyl, Reynoutria sachalinensis extract, alpha-chlorohydrin, antu, barium carbonate, bisthiosemi, brodifacoum, bromadiolone, bromethalin, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, norbormide, phosacetim, phosphorus, pindone, pyrinuron, scilliroside, sodium fluoroacetate, thallium sulfate, warfarin, 2-(2-butoxyethoxy)ethyl piperonylate, 5-(1,3-benzodioxol-5-yl)-3-hexylcyclohex-2-enone, farnesol with nerolidol, verbutin, MGK 264, piperonyl butoxide, piprotal, propyl isomer, S421, sesamex, sesasmolin, sulfoxide, anthraquinone, copper naphthenate, copper oxychloride, dicyclopentadiene, thiram, zinc naphthenate, ziram, imanin, ribavirin, chloroinconazide, mercuric oxide, thiophanate-methyl, azaconazole, bitertanol, bromuconazole, cyproconazole, difenoconazole, diniconazole, epoxiconazole, fenbuconazole, fluquinconazole, flusilazole, flutriafol, furametpyr, hexaconazole, imazalil, imibenconazole, ipconazole, metconazole, myclobutanil, paclobutrazole, pefurazoate, penconazole, prothioconazole, pyrifenox, prochloraz, propiconazole, pyrisoxazole, simeconazole, tebuconazole, tetraconazole, triadimefon, triadimenol, triflumizole, triticonazole, ancymidol, fenarimol, nuarimol, bupirimate, dimethirimol, ethirimol, dodemorph, fenpropidin, fenpropimorph, spiroxamine, tridemorph, cyprodinil, mepanipyrim, pyrimethanil, fenpiclonil, fludioxonil, benalaxyl, furalaxyl, metalaxyl, R-metalaxyl, ofurace, oxadixyl, carbendazim, debacarb, fuberidazole, thiabendazole, chlozolinate, dichlozoline, myclozoline, procymidone, vinclozoline, boscalid, carboxin, fenfuram, flutolanil, mepronil, oxycarboxin, penthiopyrad, dodine, iminoctadine, azoxystrobin, dimoxystrobin, enestroburin, thifluzamide, fenaminstrobin, flufenoxystrobin, fluoxastrobin, kresoxim-methyl, metominostrobin, trifloxystrobin, orysastrobin, picoxystrobin, pyraclostrobin, pyrametostrobin, pyraoxystrobin, ferbam, mancozeb, maneb, metiram, propineb, zineb, captafol, captan, fluoroimide, folpet, tolylfluanid, bordeaux mixture, copper oxide, mancopper, oxine-copper, nitrothal-isopropyl, edifenphos, iprobenphos, phosdiphen, tolclofos-methyl, anilazine, benthiavalicarb, blasticidin-S, chloroneb, chlorothalonil, cyflufenamid, cymoxanil, cyclobutrifluram, diclocymet, diclomezine, dicloran, diethofencarb, dimethomorph, flumorph, dithianon, ethaboxam, etridiazole, famoxadone, fenamidone, fenoxanil, ferimzone, fluazinam, flumetylsulforim, fluopicolide, fluoxytioconazole, flusulfamide, fluxapyroxad, fenhexamid, fosetyl-aluminium, hymexazol, iprovalicarb, cyazofamid, methasulfocarb, metrafenone, pencycuron, phthalide, polyoxins, propamocarb, pyribencarb, proquinazid, pyroquilon, pyriofenone, quinoxyfen, quintozene, tiadinil, triazoxide, tricyclazole, triforine, validamycin, valifenalate, zoxamide, mandipropamid, flubeneteram, isopyrazam, sedaxane, pydiflumetofen, 3-difluoromethyl-1-methyl-1H-pyrazole-4-carboxylic acid (3′,4′,5′-trifluoro-biphenyl-2-yl)-amide, isoflucypram, isotianil, dipymetitrone, 6-ethyl-5,7-dioxo-pyrrolo[4,5][1,4]dithiino [1,2-c]isothiazole-3-carbonitrile, 2-(difluoromethyl)-N-[3-ethyl-1,1-dimethyl-indan-4-yl]pyridine-3-carboxamide, 4-(2,6-difluorophenyl)-6-methyl-5-phenyl-pyridazine-3-carbonitrile, (R)-3-(difluoromethyl)-1-methyl-N-[1,1,3-trimethylindan-4-yl]pyrazole-4-carboxamide, 4-(2-bromo-4-fluoro-phenyl)-N-(2-chloro-6-fluoro-phenyl)-2,5-dimethyl-pyrazol-3-amine, 4-(2-bromo-4-fluorophenyl)-N-(2-chloro-6-fluorophenyl)-1, 3-dimethyl-1H-pyrazol-5-amine, fluindapyr, coumethoxystrobin (jiaxiangjunzhi), lvbenmixianan, dichlobentiazox, mandestrobin, 3-(4,4-difluoro-3,4-dihydro-3,3-dimethylisoquinolin-1-yl) quinolone, 2-[2-fluoro-6-[(8-fluoro-2-methyl-3-quinolyl)oxy]phenyl]propan-2-ol, oxathiapiprolin, tert-butyl N-[6-[[[(1-methyltetrazol-5-yl)-phenyl-methylene]amino]oxymethyl]-2-pyridyl]carbamate, pyraziflumid, inpyrfluxam, trolprocarb, mefentrifluconazole, ipfentrifluconazole, 2-(difluoromethyl)-N-[(3R)-3-ethyl-1,1-dimethyl-indan-4-yl]pyridine-3-carboxamide, N′-(2,5-dimethyl-4-phenoxy-phenyl)-N-ethyl-N-methyl-formamidine, N′-[4-(4,5-dichlorothiazol-2-yl)oxy-2,5-dimethyl-phenyl]-N-ethyl-N-methyl-formamidine, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate, but-3-ynyl N-[6-[[(Z)-[(1-methyltetrazol-5-yl)-phenyl-methylene]amino]oxymethyl]-2-pyridyl]carbamate, methyl N-[[5-[4-(2,4-dimethylphenyl)triazol-2-yl]-2-methyl-phenyl]methyl]carbamate, 3-chloro-6-methyl-5-phenyl-4-(2,4,6-trifluorophenyl)pyridazine, pyridachlometyl, 3-(difluoromethyl)-1-methyl-N-[1,1,3-trimethylindan-4-yl]pyrazole-4-carboxamide, 1-[2-[[1-(4-chlorophenyl)pyrazol-3-yl]oxymethyl]-3-methyl-phenyl]-4-methyl-tetrazol-5-one, 1-methyl-4-[3-methyl-2-[[2-methyl-4-(3,4,5-trimethylpyrazol-1-yl)phenoxy]methyl]phenyl]tetrazol-5-one, aminopyrifen, ametoctradin, amisulbrom, penflufen, (Z,2E)-5-[1-(4-chlorophenyl)pyrazol-3-yl]oxy-2-methoxyimino-N,3-dimethyl-pent-3-enamide, florylpicoxamid, fenpicoxamid, metarylpicoxamid, tebufloquin, ipflufenoquin, quinofumelin, isofetamid, ethyl 1-[[4-[[2-(trifluoromethyl)-1,3-dioxolan-2-yl]methoxy]phenyl]methyl]pyrazole-3-carboxylate (may be prepared from the methods described in WO 2020/056090), ethyl 1-[[4-[(Z)-2-ethoxy-3,3,3-trifluoro-prop-1-enoxy]phenyl]methyl]pyrazole-3-carboxylate (may be prepared from the methods described in WO 2020/056090), methyl N-[[4-[1-(4-cyclopropyl-2,6-difluoro-phenyl)pyrazol-4-yl]-2-methyl-phenyl]methyl]carbamate (may be prepared from the methods described in WO 2020/097012), methyl N-[[4-[1-(2,6-difluoro-4-isopropyl-phenyl)pyrazol-4-yl]-2-methyl-phenyl]methyl]carbamate (may be prepared from the methods described in WO 2020/097012), 6-chloro-3-(3-cyclopropyl-2-fluoro-phenoxy)-N-[2-(2,4-dimethylphenyl)-2,2-difluoro-ethyl]-5-methyl-pyridazine-4-carboxamide (may be prepared from the methods described in WO 2020/109391), 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide (may be prepared from the methods described in WO 2020/109391), 6-chloro-3-(3-cyclopropyl-2-fluoro-phenoxy)-N-[2-(3,4-dimethylphenyl)-2,2-difluoro-ethyl]-5-methyl-pyridazine-4-carboxamide (may be prepared from the methods described in WO 2020/109391), N-[2-[2,4-dichloro-phenoxy]phenyl]-3-(difluoromethyl)-1-methyl-pyrazole-4-carboxamide, N-[2-[2-chloro-4-(trifluoromethyl)phenoxy]phenyl]-3-(difluoromethyl)-1-methyl-pyrazole-4-carboxamide, benzothiostrobin, phenamacril, 5-amino-1,3,4-thiadiazole-2-thiol zinc salt (2:1), fluopyram, flufenoxadiazam, flutianil, fluopimomide, pyrapropoyne, picarbutrazox, 2-(difluoromethyl)-N-(3-ethyl-1,1-dimethyl-indan-4-yl)pyridine-3-carboxamide, 2-(difluoromethyl)-N-((3R)-1, 1, 3-trimethylindan-4-yl)pyridine-3-carboxamide, 4-[[6-[2-(2,4-difluorophenyl)-1,1-difluoro-2-hydroxy-3-(1,2,4-triazol-1-yl)propyl]-3-pyridyl]oxy]benzonitrile, metyltetraprole, 2-(difluoromethyl)-N-((3R)-1, 1, 3-trimethylindan-4-yl)pyridine-3-carboxamide, α-(1, 1-dimethylethyl)-α-[4′-(trifluoromethoxy) [1,1′-biphenyl]-4-yl]-5-pyrimidinemethanol, fluoxapiprolin, enoxastrobin, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate (these compounds may be prepared from the methods described in WO2020/079111), methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 4-[[6-[2-(2,4-difluorophenyl)-1,1-difluoro-2-hydroxy-3-(1,2,4-triazol-1-yl)propyl]-3-pyridyl]oxy]benzonitrile, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile, trinexapac, coumoxystrobin, zhongshengmycin, thiodiazole copper, zinc thiazole, amectotractin, iprodione, seboctylamine, N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-bromo-2-methyl-6-(1-methyl-2-propoxy-ethoxy)-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, N′-[5-bromo-2-methyl-6-(1-methyl-2-propoxy-ethoxy)-3-pyridyl]-N-isopropyl-N-methyl-formamidine (these compounds may be prepared from the methods described in WO2015/155075); N′-[5-bromo-2-methyl-6-(2-propoxypropoxy)-3-pyridyl]-N-ethyl-N-methyl-formamidine (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine, N′-[4-(1-cyclopropyl-2,2,2-trifluoro-1-hydroxy-ethyl)-5-methoxy-2-methyl-phenyl]-N-isopropyl-N-methyl-formamidine (these compounds may be prepared from the methods described in WO2018/228896); N-ethyl-N′-[5-methoxy-2-methyl-4-[(2-trifluoromethyl) oxetan-2-yl]phenyl]-N-methyl-formamidine, N-ethyl-N′-[5-methoxy-2-methyl-4-[(2-trifuoromethyl)tetrahydrofuran-2-yl]phenyl]-N-methyl-formamidine (these compounds may be prepared from the methods described in WO2019/110427); N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide (these compounds may be prepared from the methods described in WO2017/153380); 1-(6,7-dimethylpyrazolo[1,5-a]pyridin-3-yl)-4,4,5-trifluoro-3,3-dimethyl-isoquinoline, 1-(6,7-dimethylpyrazolo[1,5-a]pyridin-3-yl)-4,4,6-trifluoro-3,3-dimethyl-isoquinoline, 4,4-difluoro-3,3-dimethyl-1-(6-methylpyrazolo[1,5-a]pyridin-3-yl) isoquinoline, 4,4-difluoro-3,3-dimethyl-1-(7-methylpyrazolo[1,5-a]pyridin-3-yl) isoquinoline, 1-(6-chloro-7-methyl-pyrazolo[1,5-a]pyridin-3-yl)-4,4-difluoro-3,3-dimethyl-isoquinoline (these compounds may be prepared from the methods described in WO2017/025510); 1-(4,5-dimethylbenzimidazol-1-yl)-4,4,5-trifluoro-3,3-dimethyl-isoquinoline, 1-(4,5-dimethylbenzimidazol-1-yl)-4,4-difluoro-3,3-dimethyl-isoquinoline, 6-chloro-4,4-difluoro-3,3-dimethyl-1-(4-methylbenzimidazol-1-yl) isoquinoline, 4,4-difluoro-1-(5-fluoro-4-methyl-benzimidazol-1-yl)-3,3-dimethyl-isoquinoline, 3-(4,4-difluoro-3,3-dimethyl-1-isoquinolyl)-7,8-dihydro-6H-cyclopenta[e]benzimidazole (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, N-ethyl-2-methyl-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, 3-ethyl-1-methoxy-1-[[4-[5-(trifluoromethyl)-1,2,4-oxadiazol-3-yl]phenyl]methyl]urea, N-[[4-[5-(trifluoromethyl)-1,2,4-oxadiazol-3-yl]phenyl]methyl]propanamide, 4,4-dimethyl-2-[[4-[5-(trifluoromethyl)-1,2,4-oxadiazol-3-yl]phenyl]methyl]isoxazolidin-3-one, 5,5-dimethyl-2-[[4-[5-(trifluoromethyl)-1,2,4-oxadiazol-3-yl]phenyl]methyl]isoxazolidin-3-one, ethyl 1-[[4-[5-(trifluoromethyl)-1,2,4-oxadiazol-3-yl]phenyl]methyl]pyrazole-4-carboxylate, N,N-dimethyl-1-[[4-[5-(trifluoromethyl)-1,2,4-oxadiazol-3-yl]phenyl]methyl]-1,2,4-triazol-3-amine (these compounds may be prepared from the methods described in WO 2017/055473, WO 2017/055469, WO 2017/093348 and WO 2017/118689); 2-[6-(4-chlorophenoxy)-2-(trifluoromethyl)-3-pyridyl]-1-(1,2,4-triazol-1-yl) propan-2-ol (this compound may be prepared from the methods described in WO 2017/029179); 2-[6-(4-bromophenoxy)-2-(trifluoromethyl)-3-pyridyl]-1-(1,2,4-triazol-1-yl) propan-2-ol (this compound may be prepared from the methods described in WO 2017/029179); 3-[2-(1-chlorocyclopropyl)-3-(2-fluorophenyl)-2-hydroxy-propyl]imidazole-4-carbonitrile (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile (this compound may be prepared from the methods described in WO 2016/156290); (4-phenoxyphenyl)methyl 2-amino-6-methyl-pyridine-3-carboxylate (this compound may be prepared from the methods described in WO 2014/006945); 2,6-Dimethyl-1H,5H-[1,4]dithiino[2,3-c: 5,6-c′]dipyrrole-1,3,5,7 (2H,6H)-tetrone (this compound may be prepared from the methods described in WO 2011/138281) N-methyl-4-[5-(trifluoromethyl)-1,2,4-oxadiazol-3-yl]benzenecarbothioamide; N-methyl-4-[5-(trifluoromethyl)-1,2,4-oxadiazol-3-yl]benzamide; (Z,2E)-5-[1-(2,4-dichlorophenyl)pyrazol-3-yl]oxy-2-methoxyimino-N,3-dimethyl-pent-3-enamide (this compound may be prepared from the methods described in WO 2018/153707); N′-(2-chloro-5-methyl-4-phenoxy-phenyl)-N-ethyl-N-methyl-formamidine; N′-[2-chloro-4-(2-fluorophenoxy)-5-methyl-phenyl]-N-ethyl-N-methyl-formamidine (this compound may be prepared from the methods described in WO 2016/202742); 2-(difluoromethyl)-N-[(3S)-3-ethyl-1,1-dimethyl-indan-4-yl]pyridine-3-carboxamide (this compound may be prepared from the methods described in WO 2014/095675); (5-methyl-2-pyridyl)-[4-[5-(trifluoromethyl)-(3-methylisoxazol-5-yl)-[4-[5-(trifluoromethyl)-1,2,4-oxadiazol-3-yl]phenyl]methanone, 1,2,4-oxadiazol-3-yl]phenyl]methanone (these compounds may be prepared from the methods described in WO 2017/220485); 2-oxo-N-propyl-2-[4-[5-(trifluoromethyl)-1,2,4-oxadiazol-3-yl]phenyl]acetamide (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate (this compound may be prepared from the methods described in WO 2018/158365); 2,2-difluoro-N-methyl-2-[4-[5-(trifluoromethyl)-1,2,4-oxadiazol-3-yl]phenyl]acetamide, N-[(E)-methoxyiminomethyl]-4-[5-(trifluoromethyl)-1,2,4-oxadiazol-3-yl]benzamide, N-[(Z)-methoxyiminomethyl]-4-[5-(trifluoromethyl)-1,2,4-oxadiazol-3-yl]benzamide, N-[N-methoxy-C-methyl-carbonimidoyl]-4-[5-(trifluoromethyl)-1,2,4-oxadiazol-3-yl]benzamide (these compounds may be prepared from the methods described in WO 2018/202428);
microbials including: Acinetobacter lwoffii, Acremonium alternatum, Acremonium cephalosporium, Acremonium diospyri, Acremonium obclavatum, Adoxophyes orana granulovirus (AdoxGV) (Capex®), Agrobacterium radiobacter strain K84 (Galltrol-A®), Alternaria alternate, Alternaria cassia, Alternaria destruens (Smolder®), Ampelomyces quisqualis (AQ10®), Aspergillus flavus AF36 (AF36®), Aspergillus flavus NRRL 21882 (Aflaguard®), Aspergillus spp., Aureobasidium pullulans, Azospirillum, (MicroAZ®, TAZO B®), Azotobacter, Azotobacter chroocuccum (Azotomeal®), Azotobacter cysts (Bionatural Blooming Blossoms®), Bacillus amyloliquefaciens, Bacillus cereus, Bacillus chitinosporus strain CM-1, Bacillus chitinosporus strain AQ746, Bacillus licheniformis strain HB-2 (Biostart™ formerly Rhizoboost®), Bacillus licheniformis strain 3086 (EcoGuard®, Green Releaf®), Bacillus circulans, Bacillus firmus (BioSafe®, BioNem-WP®, VOTIVO®), Bacillus firmus strain I-1582, Bacillus macerans, Bacillus marismortui, Bacillus megaterium, Bacillus mycoides strain AQ726, Bacillus papillae (Milky Spore Powder®), Bacillus pumilus spp., Bacillus pumilus strain GB34 (Yield Shield®), Bacillus pumilus strain AQ717, Bacillus pumilus strain QST 2808 (Sonata®, Ballad Plus®), Bacillus sphaericus (VectoLex®), Bacillus spp., Bacillus spp. strain AQ175, Bacillus spp. strain AQ177, Bacillus spp. strain AQ178, Bacillus subtilis strain QST 713 (CEASE®, Serenade®, Rhapsody®), Bacillus subtilis strain QST 714 (JAZZ®), Bacillus subtilis strain AQ153, Bacillus subtilis strain AQ743, Bacillus subtilis strain QST3002, Bacillus subtilis strain QST3004, Bacillus subtilis var. amyloliquefaciens strain FZB24 (Taegroß, Rhizopro®), Bacillus thuringiensis Cry 2Ae, Bacillus thuringiensis CrylAb, Bacillus thuringiensis aizawai GC 91 (Agree®), Bacillus thuringiensis israelensis (BMP123®, Aquabac®, VectoBac®), Bacillus thuringiensis kurstaki (Javelin®, Deliver®, CryMax®, Bonide®, Scutella WP®, Turilav WP®, Astuto®, Dipel WP®, Biobit®, Foray®), Bacillus thuringiensis kurstaki BMP 123 (Baritone®), Bacillus thuringiensis kurstaki HD-1 (Bioprotec-CAF/3P®), Bacillus thuringiensis strain BD #32, Bacillus thuringiensis strain AQ52, Bacillus thuringiensis var. aizawai (XenTari®, DiPel®), bacteria spp. (GROWMEND®, GROWSWEET®, Shootup®), bacteriophage of Clavipacter michiganensis (AgriPhage®), Bakflor®, Beauveria bassiana (Beaugenic®, Brocaril WP®), Beauveria bassiana GHA (Mycotrol ES®, Mycotrol O®, BotaniGuard®), Beauveria brongniartii (Engerlingspilz®, Schweizer Beauveria®, Melocont®), Beauveria spp., Botrytis cineria, Bradyrhizobium japonicum (TerraMax®), Brevibacillus brevis, Bacillus thuringiensis tenebrionis (Novodor®), BtBooster, Burkholderia cepacia (Deny®, Intercept®, Blue Circle®), Burkholderia gladii, Burkholderia gladioli, Burkholderia spp., Canadian thistle fungus (CBH Canadian Bioherbicide®), Candida butyri, Candida famata, Candida fructus, Candida glabrata, Candida guilliermondii, Candida melibiosica, Candida oleophila strain O, Candida parapsilosis, Candida pelliculosa, Candida pulcherrima, Candida reukaufii, Candida saitoana (Bio-Coat®, Biocure®), Candida sake, Candida spp., Candida tenius, Cedecea dravisae, Cellulomonas flavigena, Chaetomium cochliodes (Nova-Cide®), Chaetomium globosum (Nova-Cide®), Chromobacterium subtsugae strain PRAA4-1T (Grandevo®), Cladosporium cladosporioides, Cladosporium oxysporum, Cladosporium chlorocephalum, Cladosporium spp., Cladosporium tenuissimum, Clonostachys rosea (EndoFine®), Colletotrichum acutatum, Coniothyrium minitans (Cotans WG®), Coniothyrium spp., Cryptococcus albidus (YIELDPLUS®), Cryptococcus humicola, Cryptococcus infirmo-miniatus, Cryptococcus laurentii, Cryptophlebia leucotreta granulovirus (Cryptex®), Cupriavidus campinensis, Cydia pomonella granulovirus (CYD-X®), Cydia pomonella granulovirus (Madex®, Madex® Plus, Madex Max, Carpovirusine®), Cylindrobasidium laeve (Stumpout®), Cylindrocladium, Debaryomyces hansenii, Drechslera hawaiinensis, Enterobacter cloacae, Enterobacteriaceae, Entomophtora virulenta (Vektor®), Epicoccum nigrum, Epicoccum purpurascens, Epicoccum spp., Filobasidium floriforme, Fusarium acuminatum, Fusarium chlamydosporum, Fusarium oxysporum (Fusaclean®, Biofox C®), Fusarium proliferatum, Fusarium spp., Galactomyces geotrichum, Gliocladium catenulatum (Primastop®, Prestop®), Gliocladium roseum, Gliocladium spp. (SoilGard®), Gliocladium virens (Soilgard®), Granulovirus (Granupom®), Halobacillus halophilus, Halobacillus litoralis, Halobacillus trueperi, Halomonas spp., Halomonas subglaciescola, Halovibrio variabilis, Hanseniaspora uvarum, Helicoverpa armigera nucleopolyhedrovirus (Helicovex®), Helicoverpa zea nuclear polyhedrosis virus (Gemstar®), Isoflavone-formononetin (Myconate®), Kloeckera apiculata, Kloeckera spp., Lagenidium giganteum (Laginex®), Lecanicillium longisporum (Vertiblast®), Lecanicillium muscarium (Vertikil®), Lymantria Dispar nucleopolyhedrosis virus (Disparvirus®), Marinococcus halophilus, Meira geulakonigii, Metarhizium anisopliae (Met52®), Metarhizium anisopliae (Destruxin WP®), Metschnikowia fruticola (Shemer®), Metschnikowia pulcherrima, Microdochium dimerum (Antibot®), Micromonospora coerulea, Microsphaeropsis ochracea, Muscodor albus 620 (Muscudor®), Muscodor roseus strain A3-5, Mycorrhizae spp. (AMykor®, Root Maximizer®), Myrothecium verrucaria strain AARC-0255 (DiTera®), BROS PLUS®, Ophiostoma piliferum strain D97 (Sylvanex®), Paecilomyces farinosus, Paecilomyces fumosoroseus (PFR-97®, PreFeRal®), Paecilomyces linacinus (Biostat WP®), Paecilomyces lilacinus strain 251 (MeloCon WG®), Paenibacillus polymyxa, Pantoea agglomerans (BlightBan C9-1®), Pantoea spp., Pasteuria spp. (Econem®), Pasteuria nishizawae, Penicillium aurantiogriseum, Penicillium billai (Jumpstart®, TagTeam®), Penicillium brevicompactum, Penicillium frequentans, Penicillium griseofulvum, Penicillium purpurogenum, Penicillium spp., Penicillium viridicatum, Phlebiopsis gigantean (Rotstop®), phosphate solubilizing bacteria (Phosphomeal®), Phytophthora cryptogea, Phytophthora palmivora (Devine®), Pichia anomala, Pichia guilermondii, Pichia membranaefaciens, Pichia onychis, Pichia stipites, Pseudomonas aeruginosa, Pseudomonas aureofasciens (Spot-Less Biofungicide®), Pseudomonas cepacia, Pseudomonas chlororaphis (AtEze®), Pseudomonas corrugate, Pseudomonas fluorescens strain A506 (BlightBan A506®), Pseudomonas putida, Pseudomonas reactans, Pseudomonas spp., Pseudomonas syringae (Bio-Save®), Pseudomonas viridiflava, Pseudomons fluorescens (Zequanox®), Pseudozyma flocculosa strain PF-A22 UL (Sporodex L®), Puccinia canaliculata, Puccinia thlaspeos (Wood Warrior®), Pythium paroecandrum, Pythium oligandrum (Polygandron®, Polyversum®), Pythium periplocum, Rhanella aquatilis, Rhanella spp., Rhizobia (Dormal®, Vault®), Rhizoctonia, Rhodococcus globerulus strain AQ719, Rhodosporidium diobovatum, Rhodosporidium toruloides, Rhodotorula spp., Rhodotorula glutinis, Rhodotorula graminis, Rhodotorula mucilagnosa, Rhodotorula rubra, Saccharomyces cerevisiae, Salinococcus roseus, Sclerotinia minor, Sclerotinia minor (SARRITOR®), Scytalidium spp., Scytalidium uredinicola, Spodoptera exigua nuclear polyhedrosis virus (Spod-X®, Spexit®), Serratia marcescens, Serratia plymuthica, Serratia spp., Sordaria fimicola, Spodoptera littoralis nucleopolyhedrovirus (Littovir®), Sporobolomyces roseus, Stenotrophomonas maltophilia, Streptomyces ahygroscopicus, Streptomyces albaduncus, Streptomyces exfoliates, Streptomyces galbus, Streptomyces griseoplanus, Streptomyces griseoviridis (Mycostop®), Streptomyces lydicus (Actinovate®), Streptomyces lydicus WYEC-108 (ActinoGrow®), Streptomyces violaceus, Tilletiopsis minor, Tilletiopsis spp., Trichoderma asperellum (T34 Biocontrol®), Trichoderma gamsii (Tenet®), Trichoderma atroviride (Plantmate®), Trichoderma hamatum TH 382, Trichoderma harzianum rifai (Mycostar®), Trichoderma harzianum T-22 (Trianum-P®, PlantShield HC®, RootShield®, Trianum-G®), Trichoderma harzianum T-39 (Trichodex®), Trichoderma inhamatum, Trichoderma koningii, Trichoderma spp. LC 52 (Sentinel®), Trichoderma lignorum, Trichoderma longibrachiatum, Trichoderma polysporum (Binab T®), Trichoderma taxi, Trichoderma virens, Trichoderma virens (formerly Gliocladium virens GL-21) (SoilGuard®), Trichoderma viride, Trichoderma viride strain ICC 080 (Remedier®), Trichosporon pullulans, Trichosporon spp., Trichothecium spp., Trichothecium roseum, Typhula phacorrhiza strain 94670, Typhula phacorrhiza strain 94671, Ulocladium atrum, Ulocladium oudemansii (Botry-Zen®), Ustilago maydis, various bacteria and supplementary micronutrients (Natural II®), various fungi (Millennium Microbes®), Verticillium chlamydosporium, Verticillium lecanii (Mycotal®, Vertalec®), Vip3Aa20 (VIPtera®), Virgibaclillus marismortui, Xanthomonas campestris pv. Poae (Camperico®), Xenorhabdus bovienii, and Xenorhabdus nematophilus;

Plant extracts including: pine oil (Retenol®), azadirachtin (Plasma Neem Oil®, AzaGuard®, MeemAzal®, Molt-X®, Botanical IGR (Neemazad®, Neemix®), canola oil (Lilly Miller Vegol®), Chenopodium ambrosioides near ambrosioides (Requiem®), Chrysanthemum extract (Crisant®), extract of neem oil (Trilogy®), essentials oils of Labiatae (Botania®), extracts of clove rosemary peppermint and thyme oil (Garden insect Killer®), Glycinebetaine (Greenstim®), garlic, lemongrass oil (GreenMatch®), neem oil, Nepeta cataria (Catnip oil), Nepeta catarina, nicotine, oregano oil (MossBuster®), Pedaliaceae oil (Nematon®), pyrethrum, Quillaja saponaria (NemaQ®), Reynoutria sachalinensis (Regalia®, Sakalia®), rotenone (Eco Roten®), Rutaceae plant extract (Soleo®), soybean oil (Ortho Ecosense®), Melaleuca alternifolia extract (also called tea tree oil) (Timorex Gold®), thymus oil, AGNIQUE® MMF, BugOil®, mixture of rosemary sesame pepermint thyme and cinnamon extracts (EF 300®), mixture of clove rosemary and peppermint extract (EF 400®), mixture of clove pepermint garlic oil and mint (Soil Shot®), kaolin (Screen®), storage glucam of brown algae (Laminarin®);
pheromones including: blackheaded fireworm pheromone (3M Sprayable Blackheaded Fireworm Pheromone®), Codling Moth Pheromone (Paramount dispenser-(CM)/Isomate C-Plus®), Grape Berry Moth Pheromone (3M MEC-GBM Sprayable Pheromone®), Leafroller pheromone (3M MEC-LR Sprayable Pheromone®), Muscamone (Snip7 Fly Bait®, Starbar Premium Fly Bait®), Oriental Fruit Moth Pheromone (3M oriental fruit moth sprayable Pheromone®), Peachtree Borer Pheromone (Isomate-PR), Tomato Pinworm Pheromone (3M Sprayable Pheromone®), Entostat powder (extract from palm tree) (Exosex CM®), (E,Z,Z)-3,8,11 Tetradecatrienyl acetate, (Z,Z,E)-7,11,13-Hexadecatrienal, (E,Z)-7,9-Dodecadien-1-yl acetate, 2-Methyl-1-butanol, Calcium acetate, Scenturion®, Biolure®, Check-Mate®, Lavandulyl senecioate;
Macrobials including: Aphelinus abdominalis, Aphidius ervi (Aphelinus-System®), Acerophagus papaya, Adalia bipunctata (Adalia-System®), Adalia bipunctata (Adaline®), Adalia bipunctata (Aphidalia®), Ageniaspis citricola, Ageniaspis fuscicollis, Amblyseius andersoni (Anderline®, Andersoni-System®), Amblyseius californicus (Amblyline®, Spical®), Amblyseius cucumeris (Thripex®, Bugline cucumeris®), Amblyseius fallacis (Fallacis®), Amblyseius swirskii (Bugline Swirskii®, Swirskii-Mite®), Amblyseius womersleyi (WomerMite®), Amitus hesperidum, Anagrus atomus, Anagyrus fusciventris, Anagyrus kamali, Anagyrus loecki, Anagyrus pseudococci (Citripar®), Anicetus benefices, Anisopteromalus calandrae, Anthocoris nemoralis (Anthocoris-System®), Aphelinus abdominalis (Apheline®, Aphiline®), Aphelinus asychis, Aphidius colemani (Aphipar®), Aphidius ervi (Ervipar®), Aphidius gifuensis, Aphidius matricariae (Aphipar-M®), Aphidoletes aphidimyza (Aphidend®), Aphidoletes aphidimyza (Aphidoline®), Aphytis lingnanensis, Aphytis melinus, Aprostocetus hagenowii, Atheta coriaria (Staphyline®), Bombus spp., Bombus terrestris (Natupol Beehive®), Bombus terrestris (Beeline®, Tripol®), Cephalonomia stephanoderis, Chilocorus nigritus, Chrysoperla carnea (Chrysoline®), Chrysoperla carnea (Chrysopa®), Chrysoperla rufilabris, Cirrospilus ingenuus, Cirrospilus quadristriatus, Citrostichus phyllocnistoides, Closterocerus chamaeleon, Closterocerus spp., Coccidoxenoides perminutus (Planopar®), Coccophagus cowperi, Coccophagus lycimnia, Cotesia flavipes, Cotesia plutellae, Cryptolaemus montrouzieri (Cryptobug®, Cryptoline®), Cybocephalus nipponicus, Dacnusa sibirica, Dacnusa sibirica (Minusa®), Diglyphus isaea (Diminex®), Delphastus catalinae (Delphastus®), Delphastus pusillus, Diachasmimorpha krausii, Diachasmimorpha longicaudata, Diaparsis jucunda, Diaphorencyrtus aligarhensis, Diglyphus isaea, Diglyphus isaea (Miglyphus®, Digline®), Dacnusa sibirica (DacDigline®, Minex®), Diversinervus spp., Encarsia citrina, Encarsia formosa (Encarsia Max®, Encarline®, En-Strip®), Eretmocerus eremicus (Enermix®), Encarsia guadeloupae, Encarsia haitiensis, Episyrphus balteatus (Syrphidend®), Eretmoceris siphonini, Eretmocerus californicus, Eretmocerus eremicus (Ercal®, Eretline eR), Eretmocerus eremicus (Bemimix®), Eretmocerus hayati, Eretmocerus mundus (Bemipar®, Eretline M®), Eretmocerus siphonini, Exochomus quadripustulatus, Feltiella acarisuga (Spidend®), Feltiella acarisuga (Feltiline®), Fopius arisanus, Fopius ceratitivorus, Formononetin (Wirless Beehome®), Franklinothrips vespiformis (Vespop®), Galendromus occidentalis, Goniozus legneri, Habrobracon hebetor, Harmonia axyridis (HarmoBeetle®), Heterorhabditis spp. (Lawn Patrol®), Heterorhabditis bacteriophora (NemaShield HB®, Nemaseek®, Terranem-Nam®, Terranem®, Larvanem®, B-Green®, NemAttack®, Nematop®), Heterorhabditis megidis (Nemasys H®, BioNem HR, Exhibitline Hm®, Larvanem-M®), Hippodamia convergens, Hypoaspis aculeifer (Aculeifer-System®, Entomite-A®), Hypoaspis miles (Hypoline M®, Entomite-M®), Lbalia leucospoides, Lecanoideus floccissimus, Lemophagus errabundus, Leptomastidea abnormis, Leptomastix dactylopii (Leptopar®), Leptomastix epona, Lindorus lophanthae, Lipolexis oregmae, Lucilia caesar (Natufly®), Lysiphlebus testaceipes, Macrolophus caliginosus (Mirical-N®, Macroline C®, Mirical®), Mesoseiulus longipes, Metaphycus flavus, Metaphycus lounsburyi, Micromus angulatus (Milacewing®), Microterys flavus, Muscidifurax raptorellus and Spalangia cameroni (Biopar®), Neodryinus typhlocybae, Neoseiulus californicus, Neoseiulus cucumeris (THRYPEX®), Neoseiulus fallacis, Nesideocoris tenuis (NesidioBug®, Nesibug®), Ophyra aenescens (Biofly®), Orius insidiosus (Thripor-I®, Oriline I®), Orius laevigatus (Thripor-L®, Oriline 1®), Orius majusculus (Oriline M®), Orius strigicollis (Thripor-S®), Pauesia juniperorum, Pediobius foveolatus, Phasmarhabditis hermaphrodita (Nemaslug®), Phymastichus coffea, Phytoseiulus macropilus, Phytoseiulus persimilis (Spidex®, Phytoline P®), Podisus maculiventris (Podisus®), Pseudacteon curvatus, Pseudacteon obtusus, Pseudacteon tricuspis, Pseudaphycus maculipennis, Pseudleptomastix mexicana, Psyllaephagus pilosus, Psyttalia concolor (complex), Quadrastichus spp., Rhyzobius lophanthae, Rodolia cardinalis, Rumina decollate, Semielacher petiolatus, Sitobion avenae (Ervibank®), Steinernema carpocapsae (Nematac C®, Millenium®, BioNem C®, NemAttack®, Nemastar®, Capsanem®), Steinernema feltiae (NemaShield®, Nemasys F®, BioNem F®, Steinernema-System®, NemAttack®, Nemaplus®, Exhibitline Sf®, Scia-Rid® Entonem®), Steinernema kraussei (Nemasys L®, BioNem L®, Exhibitline Srb®), Steinernema riobrave (BioVector®, BioVektor®), Steinernema scapterisci (Nematac S®), Steinernema spp., Steinernematid spp. (Guardian Nematodes®), Stethorus punctillum (Stethorus®), Tamarixia radiate, Tetrastichus setifer, Thripobius semiluteus, Torymus sinensis, Trichogramma brassicae (Tricholine B®), Trichogramma brassicae (Tricho-Strip®), Trichogramma evanescens, Trichogramma minutum, Trichogramma ostriniae, Trichogramma platneri, Trichogramma pretiosum, Xanthopimpla stemmator;
other biologicals including: abscisic acid, bioSea®, Chondrostereum purpureum (Chontrol Paste®), Colletotrichum gloeosporioides (Collego®), Copper Octanoate (Cueva®), Delta traps (Trapline D®), Erwinia amylovora (Harpin) (ProAct®, Ni-HIBIT Gold CST®), fatty acids derived from a natural by-product of extra virgin olive oil (FLIPPER®), Ferri-phosphate (Ferramol®), Funnel traps (Trapline Y®), Gallex®, Grower's Secret®, Homo-brassonolide, Iron Phosphate (Lilly Miller Worry Free Ferramol Slug & Snail Bait®), MCP hail trap (Trapline F®), Microctonus hyperodae, Mycoleptodiscus terrestris (Des-X®), BioGain®, Aminomite®, Zenox®, Pheromone trap (Thripline Ams®), potassium bicarbonate (MilStop®), potassium salts of fatty acids (Sanova®), potassium silicate solution (Sil-Matrix®), potassium iodide+potassiumthiocyanate (Enzicur®), SuffOil-X®, Spider venom, Nosema locustae (Semaspore Organic Grasshopper Control®), Sticky traps (Trapline YF®, Rebell Amarillo®) and Traps (Takitrapline y+B®);
(1) antibacterial agents selected from the group of:
(1.1) bacteria, examples of which are Bacillus mojavensis strain R3B (Accession No. NCAIM (P) B001389) (WO 2013/034938) from Certis USA LLC; Bacillus pumilus, in particular strain BU F-33, having NRRL Accession No. 50185 (CARTISSA® from BASF, EPA Reg. No. 71840-19); Bacillus subtilis, in particular strain QST713/AQ713 (available as SERENADE OPTI or SERENADE ASO from Bayer CropScience LP, US, having NRRL Accession No. B21661, U.S. Pat. No. 6,060,051); Bacillus subtilis strain BU1814, (VELONDIS® PLUS, VELONDIS® FLEX and VELONDIS® EXTRA from BASF SE); Bacillus subtilis var. amyloliquefaciens strain FZB24 having Accession No. DSM 10271 (available from Novozymes as TAEGRO® or TAEGRO® ECO (EPA Registration No. 70127-5)); Bacillus subtilis CX-9060 from Certis USA LLC; Bacillus sp., in particular strain D747 (available as DOUBLE NICKEL® from Kumiai Chemical Industry Co., Ltd.), having Accession No. FERM BP-8234, U.S. Pat. No. 7,094,592; Paenibacillus sp. strain having Accession No. NRRL B-50972 or Accession No. NRRL B-67129, WO 2016/154297; Paenibacillus polymyxa, in particular strain AC-1 (e.g. TOPSEED® from Green Biotech Company Ltd.); Pantoea agglomerans, in particular strain E325 (Accession No. NRRL B-21856) (available as BLOOMTIME BIOLOGICAL™ FD BIOPESTICIDE from Northwest Agri Products); Pseudomonas proradix (e.g. PRORADIX® from Sourcon Padena); and (1.2) fungi, examples of which are Aureobasidium pullulans, in particular blastospores of strain DSM14940, blastospores of strain DSM 14941 or mixtures of blastospores of strains DSM14940 and DSM14941 (e.g., BOTECTOR® and BLOSSOM PROTECT® from bio-ferm, CH); Pseudozyma aphidis (as disclosed in WO2011/151819 by Yissum Research Development Company of the Hebrew University of Jerusalem); Saccharomyces cerevisiae, in particular strains CNCM No. 1-3936, CNCM No. 1-3937, CNCM No. 1-3938 or CNCM No. 1-3939 (WO 2010/086790) from Lesaffre et Compagnie, FR;
(2) biological fungicides selected from the group of:
(2.1) bacteria, examples of which are Agrobacterium radiobacter strain K84 (e.g. GALLTROL-AR from AgBioChem, CA); Agrobacterium radiobacter strain K1026 (e.g. NOGALL™ from BASF SE); Bacillus subtilis var. amyloliquefaciens strain FZB24 having Accession No. DSM 10271 (available from Novozymes as TAEGRO® or TAEGRO® ECO (EPA Registration No. 70127-5)); Bacillus amyloliquefaciens, in particular strain D747 (available as Double Nickel™ from Kumiai Chemical Industry Co., Ltd., having accession number FERM BP-8234, U.S. Pat. No. 7,094,592); Bacillus amyloliquefaciens strain F727 (also known as strain MBI110) (NRRL Accession No. B-50768, WO 2014/028521) (STARGUS® from Marrone Bio Innovations); Bacillus amyloliquefaciens strain FZB42, Accession No. DSM 23117 (available as RHIZOVITAL® from ABiTEP, DE); Bacillus amyloliquefaciens isolate B246 (e.g. AVOGREEN™ from University of Pretoria); Bacillus licheniformis, in particular strain SB3086, having Accession No. ATCC 55406, WO 2003/000051 (available as ECOGUARD® Biofungicide and GREEN RELEAF™ from Novozymes); Bacillus licheniformis FMCH001 and Bacillus subtilis FMCH002 (QUARTZO® (WG) and PRESENCE® (WP) from FMC Corporation); Bacillus methylotrophicus strain BAC-9912 (from Chinese Academy of Sciences' Institute of Applied Ecology); Bacillus mojavensis strain R3B (Accession No. NCAIM (P) B001389) (WO 2013/034938) from Certis USA LLC; Bacillus mycoides, isolate, having Accession No. B-30890 (available as BMJ TGAI® or WG and LifeGard™ from Certis USA LLC); Bacillus pumilus, in particular strain QST2808 (available as SONATA® from Bayer CropScience LP, US, having Accession No. NRRL B-30087 and described in U.S. Pat. No. 6,245,551); Bacillus pumilus, in particular strain GB34 (available as Yield Shield® from Bayer AG, DE); Bacillus pumilus, in particular strain BU F-33, having NRRL Accession No. 50185 (available as part of the CARTISSA product from BASF, EPA Reg. No. 71840-19); Bacillus subtilis, in particular strain QST713/AQ713 (available as SERENADE OPTI or SERENADE ASO from Bayer CropScience LP, US, having NRRL Accession No. B21661 and described in U.S. Pat. No. 6,060,051); Bacillus subtilis Y1336 (available as BIOBAC® WP from Bion-Tech, Taiwan, registered as a biological fungicide in Taiwan under Registration Nos. 4764, 5454, 5096 and 5277); Bacillus subtilis strain MBI 600 (available as SUBTILEX from BASF SE), having Accession Number NRRL B-50595, U.S. Pat. No. 5,061,495; Bacillus subtilis strain GB03 (available as Kodiak® from Bayer AG, DE); Bacillus subtilis strain BU1814, (available as VELONDIS® PLUS, VELONDIS® FLEX and VELONDIS® EXTRA from BASF SE); Bacillus subtilis CX-9060 from Certis USA LLC; Bacillus subtilis KTSB strain (FOLIACTIVE® from Donaghys); Bacillus subtilis IAB/BS03 (AVIV™ from STK Bio-Ag Technologies, PORTENTO® from Idai Nature); Bacillus subtilis strain Y1336 (available as BIOBAC® WP from Bion-Tech, Taiwan, registered as a biological fungicide in Taiwan under Registration Nos. 4764, 5454, 5096 and 5277); Paenibacillus epiphyticus (WO 2016/020371) from BASF SE; Paenibacillus polymyxa ssp. plantarum (WO 2016/020371) from BASF SE; Paenibacillus sp. strain having Accession No. NRRL B-50972 or Accession No. NRRL B-67129, WO 2016/154297; Pseudomonas chlororaphis strain AFS009, having Accession No. NRRL B-50897, WO 2017/019448 (e.g., HOWLER™ and ZIO® from AgBiome Innovations, US); Pseudomonas chlororaphis, in particular strain MA342 (e.g. CEDOMON®, CERALL®, and CEDRESS® by Bioagri and Koppert); Pseudomonas fluorescens strain A506 (e.g. BLIGHTBAN® A506 by NuFarm); Pseudomonas proradix (e.g. PRORADIX® from Sourcon Padena); Streptomyces griseoviridis strain K61 (also known as Streptomyces galbus strain K61) (Accession No. DSM 7206) (MYCOSTOP® from Verdera, PREFENCE® from BioWorks, cf. Crop Protection 2006, 25, 468-475); Streptomyces lydicus strain WYEC108 (also known as Streptomyces lydicus strain WYCD108US) (ACTINO-IRON® and ACTINOVATE® from Novozymes); and
(2.2) fungi, examples of which are Ampelomyces quisqualis, in particular strain AQ 10 (e.g. AQ 10® by IntrachemBio Italia); Ampelomyces quisqualis strain AQ10, having Accession No. CNCM 1-807 (e.g., AQ 10® by IntrachemBio Italia); Aspergillus flavus strain NRRL 21882 (products known as AFLA-GUARD® from Syngenta/ChemChina); Aureobasidium pullulans, in particular blastospores of strain DSM14940; Aureobasidium pullulans, in particular blastospores of strain DSM 14941; Aureobasidium pullulans, in particular mixtures of blastospores of strains DSM14940 and DSM 14941 (e.g. Botector® by bio-ferm, CH); Chaetomium cupreum (Accession No. CABI 353812) (e.g. BIOKUPRUM™ by AgriLife); Chaetomium globosum (available as RIVADIOM® by Rivale); Cladosporium cladosporioides, strain H39, having Accession No. CBS122244, US 2010/0291039 (by Stichting Dienst Landbouwkundig Onderzoek); Coniothyrium minitans, in particular strain CON/M/91-8 (Accession No. DSM9660, e.g. Contans® from Bayer CropScience Biologics GmbH); Cryptococcus flavescens, strain 3C (NRRL Y-50378), (B2.2.99); Dactylaria candida; Dilophosphora alopecuri (available as TWIST FUNGUS®); Fusarium oxysporum, strain Fo47 (available as FUSACLEAN® by Natural Plant Protection); Gliocladium catenulatum (Synonym: Clonostachys rosea f. catenulate) strain J1446 (e.g. Prestop® by Lallemand); Gliocladium roseum (also known as Clonostachys rosea f rosea), in particular strain 321U from Adjuvants Plus, strain ACM941 as disclosed in Xue A. G. (Efficacy of Clonostachys rosea strain ACM941 and fungicide seed treatments for controlling the root tot complex of field pea, Can Jour Plant Sci 2003, 83 (3): 519-524), or strain IK726 (Jensen D F, et al. Development of a biocontrol agent for plant disease control with special emphasis on the near commercial fungal antagonist Clonostachys rosea strain ‘IK726’, Australasian Plant Pathol. 2007, 36 (2): 95-101); Lecanicillium lecanii (formerly known as Verticillium lecanii) conidia of strain KV01 (e.g. Vertalec® by Koppert/Arysta); Metschnikowia fructicola, in particular strain NRRL Y-30752, (B2.2.3); Microsphaeropsis ochracea; Muscodor roseus, in particular strain A3-5 (Accession No. NRRL 30548); Penicillium steckii (DSM 27859, WO 2015/067800) from BASF SE; Penicillium vermiculatum; Phlebiopsis gigantea strain VRA 1992 (ROTSTOP® C from Danstar Ferment); Pichia anomala, strain WRL-076 (NRRL Y-30842), U.S. Pat. No. 7,579,183; Pseudozyma flocculosa, strain PF-A22 UL (available as SPORODEX® L by Plant Products Co., CA); Saccharomyces cerevisiae, in particular strain LASO2 (from Agro-Levures et Dérivés), strain LAS117 cell walls (CEREVISANE® from Lesaffre, ROMEO® from BASF SE), strains CNCM No. 1-3936, CNCM No. 1-3937, CNCM No. 1-3938, CNCM No. 1-3939 (WO 2010/086790) from Lesaffre et Compagnie, FR; Simplicillium lanosoniveum; Talaromyces flavus, strain V117b; Trichoderma asperelloides JM41R (Accession No. NRRL B-50759) (TRICHO PLUS® from BASF SE); Trichoderma asperellum, in particular, strain kd (e.g. T-Gro from Andermatt Biocontrol); Trichoderma asperellum, in particular strain SKT-1, having Accession No. FERM P-16510 (e.g. ECO-HOPE® from Kumiai Chemical Industry), strain T34 (e.g. T34 Biocontrol by Biocontrol Technologies S. L., ES) or strain ICC 012 from Isagro; Trichoderma atroviride, in particular strain SC1 (Accession No. CBS 122089, WO 2009/116106 and U.S. Pat. No. 8,431,120 (from Bi-PA)), strain 77B (T77 from Andermatt Biocontrol) or strain LU132 (e.g. Sentinel from Agrimm Technologies Limited); Trichoderma atroviride, strain CNCM 1-1237 (e.g. Esquive® WP from Agrauxine, FR); Trichoderma atroviride, strain no. V08/002387; Trichoderma atroviride, strain NMI no. V08/002388; Trichoderma atroviride, strain NMI no. V08/002389; Trichoderma atroviride, strain NMI no. V08/002390; Trichoderma atroviride, strain LC52 (e.g. Tenet by Agrimm Technologies Limited); Trichoderma atroviride, strain ATCC 20476 (IMI 206040); Trichoderma atroviride, strain T11 (IMI352941/CECT20498); Trichoderma atroviride, strain SKT-1 (FERM P-16510), JP Patent Publication (Kokai) 11-253151 A; Trichoderma atroviride, strain SKT-2 (FERM P-16511), JP Patent Publication (Kokai) 11-253151 A; Trichoderma atroviride, strain SKT-3 (FERM P-17021), JP Patent Publication (Kokai) 11-253151 A; Trichoderma fertile (e.g. product TrichoPlus from BASF); Trichoderma gamsii (formerly T. viride), strain ICC080 (IMI CC 392151 CABI, e.g. BioDerma by AGROBIOSOL DE MEXICO, S. A. DE C.V.); Trichoderma gamsii (formerly T. viride), strain ICC 080 (IMI CC 392151 CABI) (available as BIODERMA® by AGROBIOSOL DE MEXICO, S.A. DE C.V.); Trichoderma harmatum; Trichoderma harmatum, having Accession No. ATCC 28012; Trichoderma harzianum strain T-22 (e.g. Trianum-P from Andermatt Biocontrol or Koppert) or strain Cepa SimbT5 (from Simbiose Agro); Trichoderma harzianum; Trichoderma harzianum rifai T39 (e.g. Trichodex® from Makhteshim, US); Trichoderma harzianum, strain ITEM 908 (e.g. Trianum-P from Koppert); Trichoderma harzianum, strain TH35 (e.g. Root-Pro by Mycontrol); Trichoderma harzianum, strain DB 103 (available as T-GRO® 7456 by Dagutat Biolab); Trichoderma polysporum, strain IMI 206039 (e.g. Binab TF WP by BINAB Bio-Innovation AB, Sweden); Trichoderma stromaticum, having Accession No. Ts3550 (e.g. Tricovab by CEPLAC, Brazil); Trichoderma virens (also known as Gliocladium virens), in particular strain GL-21 (e.g. SoilGard by Certis, US); Trichoderma virens strain G-41, formerly known as Gliocladium virens (Accession No. ATCC 20906) (e.g., ROOTSHIELD® PLUS WP and TURFSHIELD® PLUS WP from BioWorks, US); Trichoderma viride, strain TV1 (e.g. Trianum-P by Koppert); Trichoderma viride, in particular strain B35 (Pietr et al., 1993, Zesz. Nauk. A R w Szczecinie 161:125-137); mixtures of Trichoderma asperellum strain ICC 012 (also known as Trichoderma harzianum ICC012), having Accession No. CABI CC IMI 392716 and Trichoderma gamsii (formerly T. viride) strain ICC 080, having Accession No. IMI 392151 (e.g., BIO-TAM™ from Isagro USA, Inc. or BIODERMA® by Agrobiosol de Mexico, S.A. de C.V.); Ulocladium oudemansii strain U3, having Accession No. NM 99/06216 (e.g., BOTRY-ZEN® by Botry-Zen Ltd, New Zealand and BOTRYSTOP® from BioWorks, Inc.); Verticillium albo-atrum (formerly V. dahliae), strain WCS850 having Accession No. WCS850, deposited at the Central Bureau for Fungi Cultures (e.g., DUTCH TRIG® by Tree Care Innovations); Verticillium chlamydosporium;
(3) biological control agents having an effect for improving plant growth and/or plant health selected from the group of:
(3.1) bacteria, examples of which are Azospirillum brasilense (e.g., VIGOR® from KALO, Inc.); Azospirillum lipoferum (e.g., VERTEX-IF™ from TerraMax, Inc.); Azorhizobium caulinodans, in particular strain ZB-SK-5; Azotobacter chroococcum, in particular strain H23; Azotobacter vinelandii, in particular strain ATCC 12837; a mixture of Azotobacter vinelandii and Clostridium pasteurianum (available as INVIGORATE® from Agrinos); Bacillus amyloliquefaciens pm414 (LOLI-PEPTAR from Biofilm Crop Protection); Bacillus amyloliquefaciens SB3281 (ATCC #PTA-7542, WO 2017/205258); Bacillus amyloliquefaciens TJ1000 (available as QUIKROOTS® from Novozymes); Bacillus amyloliquefaciens, in particular strain IN937a; Bacillus amyloliquefaciens, in particular strain FZB42 (e.g. RHIZOVITAL® from ABiTEP, DE); Bacillus amyloliquefaciens BS27 (Accession No. NRRL B-5015); Bacillus cereus family member EE128 (NRRL No. B-50917); Bacillus cereus family member EE349 (NRRL No. B-50928); Bacillus cereus, in particular strain BP01 (ATCC 55675, e.g. MEPICHLOR® from Arysta Lifescience, US); Bacillus firmus, in particular strain CNMC 1-1582 (e.g. VOTIVO® from BASF SE); Bacillus mycoides BT155 (NRRL No. B-50921); Bacillus mycoides EE118 (NRRL No. B-50918); Bacillus mycoides EE141 (NRRL No. B-50916); Bacillus mycoides BT46-3 (NRRL No. B-50922); Bacillus pumilus, in particular strain QST2808 (Accession No. NRRL No. B-30087); Bacillus pumilus, in particular strain GB34 (e.g. YIELD SHIELD® from Bayer Crop Science, DE); Bacillus siamensis, in particular strain KCTC 13613T; Bacillus subtilis, in particular strain QST713/AQ713 (having NRRL Accession No. B-21661 and described in U.S. Pat. No. 6,060,051, available as SERENADER OPTI or SERENADE® ASO from Bayer CropScience LP, US); Bacillus subtilis, in particular strain AQ30002 (Accession No. NRRL No. B-50421 and described in U.S. patent application Ser. No. 13/330,576); Bacillus subtilis, in particular strain AQ30004 (NRRL No. B-50455 and described in U.S. patent application Ser. No. 13/330,576); Bacillus subtilis strain BU1814, (available as TEQUALIS® from BASF SE), Bacillus subtilis rm303 (RHIZOMAX® from Biofilm Crop Protection); Bacillus thuringiensis BT013A (NRRL No. B-50924) also known as Bacillus thuringiensis 4Q7; a mixture of Bacillus licheniformis FMCH001 and Bacillus subtilis FMCH002 (available as QUARTZO® (WG), PRESENCE® (WP) from FMC Corporation); Bacillus subtilis, in particular strain MBI 600 (e.g. SUBTILEX® from BASF SE); Bacillus tequilensis, in particular strain NII-0943; Bradyrhizobium japonicum (e.g. OPTIMIZE® from Novozymes); Delftia acidovorans, in particular strain RAY209 (e.g. BIOBOOST® from Brett Young Seeds); Mesorhizobium cicer (e.g., NODULATOR from BASF SE); Lactobacillus sp. (e.g. LACTOPLANT® from LactoPAFI); Rhizobium leguminosarium biovar viciae (e.g., NODULATOR from BASF SE); Pseudomonas proradix (e.g. PRORADIX® from Sourcon Padena); Pseudomonas aeruginosa, in particular strain PN1; Rhizobium leguminosarum, in particular bv. viceae strain Z25 (Accession No. CECT 4585); Paenibacillus polymyxa, in particular strain AC-1 (e.g. TOPSEED® from Green Biotech Company Ltd.); Serratia marcescens, in particular strain SRM (Accession No. MTCC 8708); Sinorhizobium meliloti strain NRG-185-1 (NITRAGIN® GOLD from Bayer CropScience); Thiobacillus sp. (e.g. CROPAID® from Cropaid Ltd UK); and (3.2) fungi, examples of which are Purpureocillium lilacinum (previously known as Paecilomyces lilacinus) strain 251 (AGAL 89/030550, e.g. BioAct from Bayer CropScience Biologics GmbH); Penicillium bilaii, strain ATCC 22348 (e.g. JumpStart® from Acceleron BioAg), Talaromyces flavus, strain V117b; Trichoderma atroviride strain CNCM 1-1237 (e.g. Esquive® WP from Agrauxine, FR), Trichoderma viride, e.g. strain B35 (Pietr et al., 1993, Zesz. Nauk. A R w Szczecinie 161:125-137); Trichoderma atroviride strain LC52 (also known as Trichoderma atroviride strain LU132, e.g. Sentinel from Agrimm Technologies Limited); Trichoderma atroviride strain SC1 (described in WO2009/116106); Trichoderma asperellum strain kd (e.g. T-Gro from Andermatt Biocontrol); Trichoderma asperellum strain (Eco-T from Plant Health Products, ZA), Trichoderma harzianum strain T-22 (e.g. Trianum-P from Andermatt Biocontrol or Koppert); Myrothecium verrucaria strain AARC-0255 (e.g. DiTera™ from Valent Biosciences); Penicillium bilaii strain ATCC ATCC20851; Pythium oligandrum strain M1 (ATCC 38472, e.g. Polyversum from Bioprepraty, CZ); Trichoderma virens strain GL-21 (e.g. SoilGard® from Certis, USA); Verticillium albo-atrum (formerly V. dahliae) strain WCS850 (CBS 276.92, e.g. Dutch Trig from Tree Care Innovations); Trichoderma atroviride, in particular strain no. V08/002387, strain no. NMI No. V08/002388, strain no. NMI No. V08/002389, strain no. NMI No. V08/002390; Trichoderma harzianum strain ITEM 908, Trichoderma harzianum, strain TSTh20; Trichoderma harzianum strain 1295-22; Pythium oligandrum strain DV74; Rhizopogon amylopogon (Myco-Sol from Agri-Enterprise, LLC, formerly Helena Chemical Company); Rhizopogon fulvigleba (e.g. Myco-Sol from Agri-Enterprise, LLC, formerly Helena Chemical Company); Trichoderma virens strain GI-3;
(4) insecticidally active biological control agents selected from
(4.1) bacteria, examples of which are Agrobacterium radiobacter strain K84 (Galltrol from AgBiochem Inc.); Bacillus amyloliquefaciens, in particular strain PTS-4838 (e.g. AVEO from Valent Biosciences, US); Bacillus firmus, in particular strain CNMC 1-1582 (e.g. VOTIVO® from BASF SE); Bacillus mycoides, isolate J. (e.g. BmJ from Certis USA LLC); Bacillus sphaericus, in particular Serotype H5a5b strain 2362 (strain ABTS-1743) (e.g. VECTOLEX® from Valent BioSciences, US); Bacillus thuringiensis subsp. aizawai, in particular strain ABTS-1857 (SD-1372, e.g. XENTARI® from Valent BioSciences); Bacillus thuringiensis subsp. aizawai, in particular serotype H-7 (e.g. FLORBAC® WG from Valent BioSciences, US); Bacillus thuringiensis israelensis strain BMP 144 (e.g. AQUABAC® by Becker Microbial Products IL); Bacillus thuringiensis subsp. israelensis (serotype H-14) strain AM65-52 (Accession No. ATCC 1276) (e.g. VECTOBAC® by Valent BioSciences, US); Bacillus thuringiensis subsp. aizawai strain GC-91; Bacillus thuringiensis var. Colmeri (e.g. TIANBAOBTC by Changzhou Jianghai Chemical Factory); Bacillus thuringiensis var. japonensis strain Buibui; Bacillus thuringiensis subsp. kurstaki strain BMP 123 (from Becker Microbial Products, IL, BARITONE from Bayer CropScience); Bacillus thuringiensis subsp. kurstaki strain HD-1 (e.g. DIPEL® ES from Valent BioSciences, US); Bacillus thuringiensis var. kurstaki strain EVB-113-19 (e.g., BIOPROTEC® from AEF Global); Bacillus thuringiensis subsp. kurstaki strain ABTS 351; Bacillus thuringiensis subsp. kurstaki strain PB 54; Bacillus thuringiensis subsp. kurstaki strain SA 11 (JAVELIN from Certis, US); Bacillus thuringiensis subsp. kurstaki strain SA 12 (THURICIDE from Certis, US); Bacillus thuringiensis subsp. kurstaki strain EG 2348 (LEPINOX from Certis, US); Bacillus thuringiensis subsp. kurstaki strain EG 7841 (CRYMAX from Certis, US); Bacillus thuringiensis subsp. tenebrionis strain NB 176 (SD-5428, e.g. NOVODOR® FC from BioFa DE); Brevibacillus laterosporus (LATERAL from Ecolibrium Biologicals); Burkholderia spp., in particular Burkholderia rinojensis strain A396 (also known as Burkholderia rinojensis strain MBI 305) (Accession No. NRRL B-50319; WO 2011/106491 and WO 2013/032693; e.g. MBI206 TGAI and ZELTO® from Marrone Bio Innovations); Chromobacterium subtsugae, in particular strain PRAA4-1T (e.g. MBI-203; e.g. GRANDEVOR from Marrone Bio Innovations); Lecanicillium muscarium Ve6 (MYCOTAL from Koppert); Paenibacillus popilliae (formerly Bacillus popilliae; e.g. MILKY SPORE POWDER™ or MILKY SPORE GRANULAR™ from St. Gabriel Laboratories); Pasteuria nishizawae strain Pnl (CLARIVA from Syngenta/ChemChina); Serratia entomophila (e.g. INVADER by Wrightson Seeds); Serratia marcescens, in particular strain SRM (Accession No. MTCC 8708); Trichoderma asperellum (TRICHODERMAX from Novozymes); Wolbachia pipientis ZAP strain (e.g., ZAP MALES® from MosquitoMate); and
(4.2) fungi, examples of which are Beauveria bassiana strain ATCC 74040 (e.g. NATURALIS® from Intrachem Bio Italia); Beauveria bassiana strain GHA (Accession No. ATCC74250, e.g. BOTANIGUARD® ES and MYCONTROL-O® from Laverlam International Corporation); Beauveria bassiana strain ATP02 (Accession No. DSM 24665); Isaria fumosorosea (previously known as Paecilomyces fumosoroseus strain) Apopka 97 (PREFERAL from SePRO); Metarhizium anisopliae 3213-1 (deposited under NRRL accession number 67074 disclosed in WO 2017/066094; Pioneer Hi-Bred International); Metarhizium robertsii 15013-1 (deposited under NRRL accession number 67073); Metarhizium robertsii 23013-3 (deposited under NRRL accession number 67075); Paecilomyces lilacinus strain 251 (MELOCON from Certis, US); Zoophtora radicans;
(5) Viruses selected from the group consisting of Adoxophyes orana (summer fruit tortrix) granulosis virus (GV); Cydia pomonella (codling moth) granulosis virus (GV); Helicoverpa armigera (cotton bollworm) nuclear polyhedrosis virus (NPV); Spodoptera exigua (beet armyworm) mNPV; Spodoptera frugiperda (fall armyworm) mNPV; Spodoptera littoralis (African cotton leafworm) NPV;
(6) Bacteria and fungi which can be added as ‘inoculant’ to plants or plant parts or plant organs and which, by virtue of their particular properties, promote plant growth and plant health selected from Agrobacterium spp.; Azorhizobium caulinodans; Azospirillum spp.; Azotobacter spp.; Bradyrhizobium spp.; Burkholderia spp., in particular Burkholderia cepacia (formerly known as Pseudomonas cepacia); Gigaspora spp., or Gigaspora monosporum; Glomus spp.; Laccaria spp.; LactoBacillus buchneri; Paraglomus spp.; Pisolithus tinctorus; Pseudomonas spp.; Rhizobium spp., in particular Rhizobium trifolii; Rhizopogon spp.; Scleroderma spp.; Suillus spp.; Streptomyces spp.;
(7) Plant extracts and products formed by microorganisms including proteins and secondary metabolites which can be used as biological control agents, selected from Allium sativum (NEMGUARD from Eco-Spray; BRALIC from ADAMA); Armour-Zen; Artemisia absinthium; Azadirachtin (e.g. AZATIN XL from Certis, US); Biokeeper WP; Brassicaceae extract, in particular oilseed rape powder or mustard powder; Cassia nigricans; Celastrus angulatus; Chenopodium anthelminticum; Chitin; Dryopteris filix-mas; Equisetum arvense; Fortune Aza; Fungastop; Chenopodium quinoa saponin extract from quinoa seeds (e.g. Heads Up® (Saponins of Quinoa) from Heads Up plant Protectants, CA); naturally occurring Blad polypeptide extracted from Lupin seeds (PROBLAD® from Certis EU); naturally occurring Blad polypeptide extracted from Lupin seeds (FRACTURE® from FMC); Pyrethrum/Pyrethrins; Quassia amara; Quercus; Quillaja extract (QL AGRI 35 from BASF); Reynoutria sachalinensis extract (REGALLIA, REGALIA MAXX from Marrone Bio); “Requiem™ Insecticide”; Rotenone; ryania/ryanodine; Symphytum officinale; Tanacetum vulgare; Thymol; Thymol mixed with Geraniol (CEDROZ from Eden Research); Thymol mixed with Geraniol and Eugenol (MEVALONE from Eden Research); Triact 70; TriCon; Tropaeulum majus; Melaleuca alternifolia extract (TIMOREX GOLD from STK); Urtica dioica; Veratrin; and Viscum album; and
a safener, such as benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil.

The weight ratio of the compound of formula I to compound II may preferably be from 100:1 to 1:100, from 50:1 to 1:50, from 20:1 to 1:40, from 15:1 to 1:30, from 12:1 to 1:25, from 10:1 to 1:20, from 5:1 and 1:15, from 3:1 to 1:10 or from 2:1 to 1:5.

In a preferred embodiment, the agrochemical composition can comprise at least one further fungicide, and optionally can further comprise at least one insecticide and/or at least one nematicide.

When the crystalline polymorph of the invention is combined with at least one additional fungicide, the following mixtures of the polymorph of formula I with compound II are preferred: mixtures with a compound selected from the group of substances consisting of petroleum oils, 1,1-bis(4-chlorophenyl)-2-ethoxyethanol, 2,4-dichlorophenyl benzenesulfonate, 2-fluoro-N-methyl-N-1-naphthylacetamide, 4-chlorophenyl phenyl sulfone, acetoprole, aldoxycarb, amidithion, amidothioate, amiton, amiton hydrogen oxalate, amitraz, aramite, arsenous oxide, azobenzene, azothoate, benomyl, benoxafos, benzyl benzoate, bixafen, brofenvalerate, bromocyclen, bromophos, bromopropylate, buprofezin, butocarboxim, butoxycarboxim, butylpyridaben, calcium polysulfide, camphechlor, carbanolate, carbophenothion, cymiazole, chinomethionat, chlorbenside, chlordimeform, chlordimeform hydrochloride, chlorfenethol, chlorfenson, chlorfensulfide, chlorobenzilate, chloromebuform, chloromethiuron, chloropropylate, chlorthiophos, cinerin I, cinerin II, cinerins, closantel, coumaphos, crotamiton, crotoxyphos, cufraneb, cyanthoate, DCPM, DDT, demephion, demephion-O, demephion-S, demeton-methyl, demeton-O, demeton-O-methyl, demeton-S, demeton-S-methyl, demeton-S-methylsulfon, dichlofluanid, dichlorvos, dicliphos, dienochlor, dimefox, dinex, dinex-diclexine, dinocap-4, dinocap-6, dinocton, dinopenton, dinosulfon, dinoterbon, dioxathion, diphenyl sulfone, disulfiram, DNOC, dofenapyn, doramectin, endothion, eprinomectin, ethoate-methyl, etrimfos, fenazaflor, fenbutatin oxide, fenothiocarb, fenpyrad, fenpyroximate, fenpyrazamine, fenson, fentrifanil, flubenzimine, flucycloxuron, fluenetil, fluorbenside, FMC 1137, formetanate, formetanate hydrochloride, formparanate, gamma-HCH, glyodin, halfenprox, hexadecyl cyclopropanecarboxylate, isocarbophos, jasmolin I, jasmolin II, jodfenphos, lindane, malonoben, mecarbam, mephosfolan, mesulfen, methacrifos, methyl bromide, metolcarb, mexacarbate, milbemycin oxime, mipafox, monocrotophos, morphothion, moxidectin, naled, 4-chloro-2-(2-chloro-2-methyl-propyl)-5-[(6-iodo-3-pyridyl) methoxy]pyridazin-3-one, nifluridide, nikkomycins, nitrilacarb, nitrilacarb 1:1 zinc chloride complex, omethoate, oxydeprofos, oxydisulfoton, pp′-DDT, parathion, permethrin, phenkapton, phosalone, phosfolan, phosphamidon, polychloroterpenes, polynactins, proclonol, promacyl, propoxur, prothidathion, prothoate, pyrethrin I, pyrethrin II, pyrethrins, pyridaphenthion, pyrimitate, quinalphos, quintiofos, R-1492, phosglycin, rotenone, schradan, sebufos, selamectin, sophamide, SSI-121, sulfiram, sulfluramid, sulfotep, sulfur, diflovidazin, tau-fluvalinate, TEPP, terbam, tetradifon, tetrasul, thiafenox, thiocarboxime, thiofanox, thiometon, thioquinox, thuringiensin, triamiphos, triarathene, triazophos, triazuron, trifenofos, trinactin, vamidothion, vaniliprole, bethoxazin, copper dioctanoate, copper sulfate, cybutryne, dichlone, dichlorophen, endothal, fentin, hydrated lime, nabam, quinoclamine, quinonamid, simazine, triphenyltin acetate, triphenyltin hydroxide, crufomate, piperazine, thiophanate, chloralose, fenthion, pyridin-4-amine, strychnine, 1-hydroxy-1H-pyridine-2-thione, 4-(quinoxalin-2-ylamino)benzenesulfonamide, 8-hydroxyquinoline sulfate, bronopol, copper hydroxide, cresol, dipyrithione, dodicin, fenaminosulf, formaldehyde, hydrargaphen, kasugamycin, kasugamycin hydrochloride hydrate, nickel bis(dimethyldithiocarbamate), nitrapyrin, octhilinone, oxolinic acid, oxytetracycline, potassium hydroxyquinoline sulfate, probenazole, streptomycin, streptomycin sesquisulfate, tecloftalam, thiomersal, Adoxophyes orana GV, Agrobacterium radiobacter, Amblyseius spp., Anagrapha falcifera NPV, Anagrus atomus, Aphelinus abdominalis, Aphidius colemani, Aphidoletes aphidimyza, Autographa californica NPV, Bacillus sphaericus Neide, Beauveria brongniartii, Chrysoperla carnea, Cryptolaemus montrouzieri, Cydia pomonella GV, Dacnusa sibirica, Diglyphus isaea, Encarsia formosa, Eretmocerus eremicus, Heterorhabditis bacteriophora and H. megidis, Hippodamia convergens, Leptomastix dactylopii, Macrolophus caliginosus, Mamestra brassicae NPV, Metaphycus helvolus, Metarhizium anisopliae var. acridum, Metarhizium anisopliae var. anisopliae, Neodiprion sertifer NPV and N. lecontei NPV, Orius spp., Paecilomyces fumosoroseus, Phytoseiulus persimilis, Steinernema bibionis, Steinernema carpocapsae, Steinernema feltiae, Steinernema glaseri, Steinernema riobrave, Steinernema riobravis, Steinernema scapterisci, Steinernema spp., Trichogramma spp., Typhlodromus occidentalis, Verticillium lecanii, apholate, bisazir, busulfan, dimatif, hemel, hempa, metepa, methiotepa, methyl apholate, morzid, penfluron, tepa, thiohempa, thiotepa, tretamine, uredepa, (E)-dec-5-en-1-yl acetate with (E)-dec-5-en-1-ol, (E)-tridec-4-en-1-yl acetate, (E)-6-methylhept-2-en-4-ol, (E,Z)-tetradeca-4,10-dien-1-yl acetate, (Z)-dodec-7-en-1-yl acetate, (Z)-hexadec-11-enal, (Z)-hexadec-11-en-1-yl acetate, (Z)-hexadec-13-en-11-yn-1-yl acetate, (Z)-icos-13-en-10-one, (Z)-tetradec-7-en-1-al, (Z)-tetradec-9-en-1-ol, (Z)-tetradec-9-en-1-yl acetate, (7E,9Z)-dodeca-7,9-dien-1-yl acetate, (9Z,11E)-tetradeca-9,11-dien-1-yl acetate, (9Z,12E)-tetradeca-9,12-dien-1-yl acetate, 14-methyloctadec-1-ene, 4-methylnonan-5-ol with 4-methylnonan-5-one, alpha-multistriatin, brevicomin, codlelure, codlemone, cuelure, disparlure, dodec-8-en-1-yl acetate, dodec-9-en-1-yl acetate, dodeca-8, 10-dien-1-yl acetate, dominicalure, ethyl 4-methyloctanoate, eugenol, frontalin, grandlure, grandlure I, grandlure II, grandlure III, grandlure IV, hexalure, ipsdienol, ipsenol, japonilure, lineatin, litlure, looplure, medlure, megatomoic acid, methyl eugenol, muscalure, octadeca-2,13-dien-1-yl acetate, octadeca-3,13-dien-1-yl acetate, orfralure, oryctalure, ostramone, siglure, sordidin, sulcatol, tetradec-11-en-1-yl acetate, trimedlure, trimedlure A, trimedlure B1, trimedlure B2, trimedlure C, trunc-call, 2-(octylthio)ethanol, butopyronoxyl, butoxy (polypropylene glycol), dibutyl adipate, dibutyl phthalate, dibutyl succinate, diethyltoluamide, dimethyl carbate, dimethyl phthalate, ethyl hexanediol, hexamide, methoquin-butyl, methylneodecanamide, oxamate, picaridin, 1-dichloro-1-nitroethane, 1,1-dichloro-2,2-bis(4-ethylphenyl)ethane, 1,2-dichloropropane with 1,3-dichloropropene, 1-bromo-2-chloroethane, 2,2,2-trichloro-1-(3,4-dichlorophenyl)ethyl acetate, 2,2-dichlorovinyl 2-ethylsulfinylethyl methyl phosphate, 2-(1,3-dithiolan-2-yl)phenyl dimethylcarbamate, 2-(2-butoxyethoxy)ethyl thiocyanate, 2-(4,5-dimethyl-1,3-dioxolan-2-yl)phenyl methylcarbamate, 2-(4-chloro-3,5-xylyloxy)ethanol, 2-chlorovinyl diethyl phosphate, 2-imidazolidone, 2-isovalerylindan-1,3-dione, 2-methyl(prop-2-ynyl)aminophenyl methylcarbamate, 2-thiocyanatoethyl laurate, 3-bromo-1-chloroprop-1-ene, 3-methyl-1-phenylpyrazol-5-yl dimethylcarbamate, 4-methyl(prop-2-ynyl)amino-3,5-xylyl methylcarbamate, 5,5-dimethyl-3-oxocyclohex-1-enyl dimethylcarbamate, acethion, acrylonitrile, aldrin, allosamidin, allyxycarb, alpha-ecdysone, aluminium phosphide, aminocarb, anabasine, athidathion, azamethiphos, Bacillus thuringiensis delta endotoxins, barium hexafluorosilicate, barium polysulfide, barthrin, Bayer 22/190, Bayer 22408, beta-cyfluthrin, beta-cypermethrin, bioethanomethrin, biopermethrin, bis(2-chloroethyl)ether, borax, bromfenvinfos, bromo-DDT, bufencarb, butacarb, butathiofos, butonate, calcium arsenate, calcium cyanide, carbon disulfide, carbon tetrachloride, cartap hydrochloride, cevadine, chlorbicyclen, chlordane, chlordecone, chloroform, chloropicrin, chlorphoxim, chlorprazophos, cis-resmethrin, cismethrin, clocythrin, copper acetoarsenite, copper arsenate, copper oleate, coumithoate, cryolite, CS 708, cyanofenphos, cyanophos, cyclethrin, cythioate, d-tetramethrin, DAEP, dazomet, decarbofuran, diamidafos, dicapthon, dichlofenthion, dicresyl, dicyclanil, dieldrin, diethyl 5-methylpyrazol-3-yl phosphate, dilor, dimefluthrin, dimetan, dimethrin, dimethylvinphos, dimetilan, dinoprop, dinosam, dinoseb, diofenolan, dioxabenzofos, dithicrofos, DSP, ecdysterone, EI 1642, EMPC, EPBP, etaphos, ethiofencarb, ethyl formate, ethylene dibromide, ethylene dichloride, ethylene oxide, EXD, fenchlorphos, fenethacarb, fenitrothion, fenoxacrim, fenpirithrin, fensulfothion, fenthion-ethyl, flucofuron, fosmethilan, fospirate, fosthietan, furathiocarb, furethrin, guazatine, guazatine acetates, sodium tetrathiocarbonate, halfenprox, HCH, HEOD, heptachlor, heterophos, HHDN, hydrogen cyanide, hyquincarb, IPSP, isazofos, isobenzan, isodrin, isofenphos, isolane, isoprothiolane, isoxathion, juvenile hormone I, juvenile hormone II, juvenile hormone III, kelevan, kinoprene, lead arsenate, leptophos, lirimfos, lythidathion, m-cumenyl methylcarbamate, magnesium phosphide, mazidox, mecarphon, menazon, mercurous chloride, mesulfenfos, metam, metam-potassium, metam-sodium, methanesulfonyl fluoride, methocrotophos, methoprene, methothrin, methoxychlor, methyl isothiocyanate, methylchloroform, methylene chloride, metoxadiazone, mirex, naftalofos, naphthalene, NC-170, nicotine, nicotine sulfate, nithiazine, nornicotine, O-5-dichloro-4-iodophenyl O-ethyl ethylphosphonothioate, O,O-diethyl O-4-methyl-2-oxo-2H-chromen-7-yl phosphorothioate, O,O-diethyl O-6-methyl-2-propylpyrimidin-4-yl phosphorothioate, O,O,O′,O′-tetrapropyl dithiopyrophosphate, oleic acid, para-dichlorobenzene, parathion-methyl, pentachlorophenol, pentachlorophenyl laurate, PH 60-38, phenkapton, phosnichlor, phosphine, phoxim-methyl, pirimetaphos, polychlorodicyclopentadiene isomers, potassium arsenite, potassium thiocyanate, precocene I, precocene II, precocene III, primidophos, profluthrin, promecarb, prothiofos, pyrazophos, pyresmethrin, quassia, quinalphos-methyl, quinothion, rafoxanide, resmethrin, rotenone, kadethrin, ryania, ryanodine, sabadilla, schradan, sebufos, SI-0009, thiapronil, sodium arsenite, sodium cyanide, sodium fluoride, sodium hexafluorosilicate, sodium pentachlorophenoxide, sodium selenate, sodium thiocyanate, sulcofuron, sulcofuron-sodium, sulfuryl fluoride, sulprofos, tar oils, tazimcarb, TDE, tebupirimfos, temephos, terallethrin, tetrachloroethane, thicrofos, thiocyclam, thiocyclam hydrogen oxalate, thionazin, thiosultap, thiosultap-sodium, tralomethrin, transpermethrin, triazamate, trichlormetaphos-3, trichloronat, trimethacarb, tolprocarb, triclopyricarb, triprene, veratridine, veratrine, XMC, zetamethrin, zinc phosphide, zolaprofos, meperfluthrin, tetramethylfluthrin, bis(tributyltin) oxide, bromoacetamide, ferric phosphate, niclosamide-olamine, tributyltin oxide, pyrimorph, trifenmorph, 1,2-dibromo-3-chloropropane, 1,3-dichloropropene, 3,4-dichlorotetrahydrothiophene 1,1-dioxide, 3-(4-chlorophenyl)-5-methylrhodanine, 5-methyl-6-thioxo-1,3,5-thiadiazinan-3-ylacetic acid, 6-isopentenylaminopurine, anisiflupurin, benclothiaz, cytokinins, DCIP, furfural, isamidofos, kinetin, Myrothecium verrucaria composition, tetrachlorothiophene, xylenols, zeatin, potassium ethylxanthate, acibenzolar, acibenzolar-S-methyl, Reynoutria sachalinensis extract, alpha-chlorohydrin, antu, barium carbonate, bisthiosemi, brodifacoum, bromadiolone, bromethalin, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, norbormide, phosacetim, phosphorus, pindone, pyrinuron, scilliroside, sodium fluoroacetate, thallium sulfate, warfarin, 2-(2-butoxyethoxy)ethyl piperonylate, 5-(1,3-benzodioxol-5-yl)-3-hexylcyclohex-2-enone, farnesol with nerolidol, verbutin, MGK 264, piperonyl butoxide, piprotal, propyl isomer, S421, sesamex, sesasmolin, sulfoxide, anthraquinone, copper naphthenate, copper oxychloride, dicyclopentadiene, thiram, zinc naphthenate, ziram, imanin, ribavirin, chloroinconazide, mercuric oxide, thiophanate-methyl, azaconazole, bitertanol, bromuconazole, cyproconazole, difenoconazole, diniconazole, epoxiconazole, fenbuconazole, fluquinconazole, flusilazole, flutriafol, furametpyr, hexaconazole, imazalil, imibenconazole, ipconazole, metconazole, myclobutanil, paclobutrazole, pefurazoate, penconazole, prothioconazole, pyrifenox, prochloraz, propiconazole, pyrisoxazole, simeconazole, tebuconazole, tetraconazole, triadimefon, triadimenol, triflumizole, triticonazole, ancymidol, fenarimol, nuarimol, bupirimate, dimethirimol, ethirimol, dodemorph, fenpropidin, fenpropimorph, spiroxamine, tridemorph, cyprodinil, mepanipyrim, pyrimethanil, fenpiclonil, fludioxonil, benalaxyl, furalaxyl, metalaxyl, R-metalaxyl, ofurace, oxadixyl, carbendazim, debacarb, fuberidazole, thiabendazole, chlozolinate, dichlozoline, myclozoline, procymidone, vinclozoline, boscalid, carboxin, fenfuram, flutolanil, mepronil, oxycarboxin, penthiopyrad, thifluzamide, dodine, iminoctadine, azoxystrobin, dimoxystrobin, enestroburin, fenaminstrobin, flufenoxystrobin, fluoxastrobin, kresoxim-methyl, metominostrobin, trifloxystrobin, orysastrobin, picoxystrobin, pyraclostrobin, pyrametostrobin, pyraoxystrobin, ferbam, mancozeb, maneb, metiram, propineb, zineb, captafol, captan, fluoroimide, folpet, tolylfluanid, bordeaux mixture, copper oxide, mancopper, oxine-copper, nitrothal-isopropyl, edifenphos, iprobenphos, phosdiphen, tolclofos-methyl, anilazine, benthiavalicarb, blasticidin-S, chloroneb, chlorothalonil, cyflufenamid, cymoxanil, cyclobutrifluram, diclocymet, diclomezine, dicloran, diethofencarb, dimethomorph, flumorph, dithianon, ethaboxam, etridiazole, famoxadone, fenamidone, fenoxanil, ferimzone, fluazinam, flumetylsulforim, fluopicolide, fluoxytioconazole, flusulfamide, fluxapyroxad, fenhexamid, fosetyl-aluminium, hymexazol, iprovalicarb, cyazofamid, methasulfocarb, metrafenone, pencycuron, phthalide, polyoxins, propamocarb, pyribencarb, proquinazid, pyroquilon, pyriofenone, quinoxyfen, quintozene, tiadinil, triazoxide, tricyclazole, triforine, validamycin, valifenalate, zoxamide, mandipropamid, flubeneteram, isopyrazam, sedaxane, pydiflumetofen, 3-difluoromethyl-1-methyl-1H-pyrazole-4-carboxylic acid (3′,4′,5′-trifluoro-biphenyl-2-yl)-amide, isoflucypram, isotianil, dipymetitrone, 6-ethyl-5,7-dioxo-pyrrolo[4,5][1,4]dithiino [1,2-c]isothiazole-3-carbonitrile, 2-(difluoromethyl)-N-[3-ethyl-1,1-dimethyl-indan-4-yl]pyridine-3-carboxamide, 4-(2,6-difluorophenyl)-6-methyl-5-phenyl-pyridazine-3-carbonitrile, (R)-3-(difluoromethyl)-1-methyl-N-[1,1,3-trimethylindan-4-yl]pyrazole-4-carboxamide, 4-(2-bromo-4-fluoro-phenyl)-N-(2-chloro-6-fluoro-phenyl)-2,5-dimethyl-pyrazol-3-amine, 4-(2-bromo-4-fluorophenyl)-N-(2-chloro-6-fluorophenyl)-1, 3-dimethyl-1H-pyrazol-5-amine, fluindapyr, coumethoxystrobin (jiaxiangjunzhi), lvbenmixianan, dichlobentiazox, mandestrobin, 3-(4,4-difluoro-3,4-dihydro-3,3-dimethylisoquinolin-1-yl) quinolone, 2-[2-fluoro-6-[(8-fluoro-2-methyl-3-quinolyl)oxy]phenyl]propan-2-ol, oxathiapiprolin, tert-butyl N-[6-[[[(1-methyltetrazol-5-yl)-phenyl-methylene]amino]oxymethyl]-2-pyridyl]carbamate, pyraziflumid, inpyrfluxam, trolprocarb, mefentrifluconazole, ipfentrifluconazole, 2-(difluoromethyl)-N-[(3R)-3-ethyl-1,1-dimethyl-indan-4-yl]pyridine-3-carboxamide, N′-(2,5-dimethyl-4-phenoxy-phenyl)-N-ethyl-N-methyl-formamidine, N′-[4-(4,5-dichlorothiazol-2-yl)oxy-2,5-dimethyl-phenyl]-N-ethyl-N-methyl-formamidine, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl]methanesulfonate, but-3-ynyl N-[6-[[(Z)-[(1-methyltetrazol-5-yl)-phenyl-methylene]amino]oxymethyl]-2-pyridyl]carbamate, methyl N-[[5-[4-(2,4-dimethylphenyl)triazol-2-yl]-2-methyl-phenyl]methyl]carbamate, 3-chloro-6-methyl-5-phenyl-4-(2,4,6-trifluorophenyl)pyridazine, pyridachlometyl, 3-(difluoromethyl)-1-methyl-N-[1,1,3-trimethylindan-4-yl]pyrazole-4-carboxamide, 1-[2-[[1-(4-chlorophenyl)pyrazol-3-yl]oxymethyl]-3-methyl-phenyl]-4-methyl-tetrazol-5-one, 1-methyl-4-[3-methyl-2-[[2-methyl-4-(3,4,5-trimethylpyrazol-1-yl)phenoxy]methyl]phenyl]tetrazol-5-one, aminopyrifen, ametoctradin, amisulbrom, penflufen, (Z,2E)-5-[1-(4-chlorophenyl)pyrazol-3-yl]oxy-2-methoxyimino-N,3-dimethyl-pent-3-enamide, florylpicoxamid, fenpicoxamid, metarylpicoxamid, tebufloquin, ipflufenoquin, quinofumelin, isofetamid, ethyl 1-[[4-[[2-(trifluoromethyl)-1,3-dioxolan-2-yl]methoxy]phenyl]methyl]pyrazole-3-carboxylate (may be prepared from the methods described in WO 2020/056090), ethyl 1-[[4-[(Z)-2-ethoxy-3,3,3-trifluoro-prop-1-enoxy]phenyl]methyl]pyrazole-3-carboxylate (may be prepared from the methods described in WO 2020/056090), methyl N-[[4-[1-(4-cyclopropyl-2,6-difluoro-phenyl)pyrazol-4-yl]-2-methyl-phenyl]methyl]carbamate (may be prepared from the methods described in WO 2020/097012), methyl N-[[4-[1-(2,6-difluoro-4-isopropyl-phenyl)pyrazol-4-yl]-2-methyl-phenyl]methyl]carbamate (may be prepared from the methods described in WO 2020/097012), 6-chloro-3-(3-cyclopropyl-2-fluoro-phenoxy)-N-[2-(2,4-dimethylphenyl)-2,2-difluoro-ethyl]-5-methyl-pyridazine-4-carboxamide (may be prepared from the methods described in WO 2020/109391), 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide (may be prepared from the methods described in WO 2020/109391), 6-chloro-3-(3-cyclopropyl-2-fluoro-phenoxy)-N-[2-(3,4-dimethylphenyl)-2,2-difluoro-ethyl]-5-methyl-pyridazine-4-carboxamide (may be prepared from the methods described in WO 2020/109391), N-[2-[2,4-dichloro-phenoxy]phenyl]-3-(difluoromethyl)-1-methyl-pyrazole-4-carboxamide, N-[2-[2-chloro-4-(trifluoromethyl)phenoxy]phenyl]-3-(difluoromethyl)-1-methyl-pyrazole-4-carboxamide, benzothiostrobin, phenamacril, 5-amino-1,3,4-thiadiazole-2-thiol zinc salt (2:1), fluopyram, flufenoxadiazam, flutianil, fluopimomide, pyrapropoyne, picarbutrazox, 2-(difluoromethyl)-N-(3-ethyl-1,1-dimethyl-indan-4-yl)pyridine-3-carboxamide, 2-(difluoromethyl)-N-((3R)-1, 1, 3-trimethylindan-4-yl)pyridine-3-carboxamide, 4-[[6-[2-(2,4-difluorophenyl)-1,1-difluoro-2-hydroxy-3-(1,2,4-triazol-1-yl)propyl]-3-pyridyl]oxy]benzonitrile, metyltetraprole, 2-(difluoromethyl)-N-((3R)-1, 1, 3-trimethylindan-4-yl)pyridine-3-carboxamide, α-(1,1-dimethylethyl)-α-[4′-(trifluoromethoxy) [1,1′-biphenyl]-4-yl]-5-pyrimidinemethanol, fluoxapiprolin, enoxastrobin, methyl (Z)-3-methoxy-2-[2-methyl-5-[4-(trifluoromethyl)triazol-2-yl]phenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl)pyrazol-1-yl]phenoxy]prop-2-enoate (these compounds may be prepared from the methods described in WO2020/079111), methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 4-[[6-[2-(2,4-difluorophenyl)-1,1-difluoro-2-hydroxy-3-(1,2,4-triazol-1-yl)propyl]-3-pyridyl]oxy]benzonitrile, 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile, trinexapac, coumoxystrobin, zhongshengmycin, thiodiazole copper, zinc thiazole, amectotractin, iprodione, seboctylamine, N′-[5-bromo-2-methyl-6-[(1S)-1-methyl-2-propoxy-ethoxy]-3-N′-[5-bromo-2-methyl-6-[(1R)-1-methyl-2-pyridyl]-N-ethyl-N-methyl-formamidine, propoxy-ethoxy]-3-pyridyl]-N-ethyl-N-methyl-formamidine, N′-[5-bromo-2-methyl-6-(1-methyl-2-propoxy-ethoxy)-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, N′-[5-bromo-2-methyl-6-(1-methyl-2-propoxy-ethoxy)-3-pyridyl]-N-isopropyl-N-methyl-formamidine (these compounds may be prepared from the methods described in WO2015/155075); N′-[5-bromo-2-methyl-6-(2-propoxypropoxy)-3-pyridyl]-N-ethyl-N-methyl-formamidine (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine, N′-[4-(1-cyclopropyl-2,2,2-trifluoro-1-hydroxy-ethyl)-5-methoxy-2-methyl-phenyl]-N-isopropyl-N-methyl-formamidine (these compounds may be prepared from the methods described in WO2018/228896); N-ethyl-N′-[5-methoxy-2-methyl-4-[(2-trifluoromethyl) oxetan-2-yl]phenyl]-N-methyl-formamidine, N-ethyl-N′-[5-methoxy-2-methyl-4-[(2-trifuoromethyl)tetrahydrofuran-2-yl]phenyl]-N-methyl-formamidine (these compounds may be prepared from the methods described in WO2019/110427); N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide (these compounds may be prepared from the methods described in WO2017/153380); 1-(6,7-dimethylpyrazolo[1,5-a]pyridin-3-yl)-4,4,5-trifluoro-3,3-dimethyl-isoquinoline, 1-(6,7-dimethylpyrazolo[1,5-a]pyridin-3-yl)-4,4,6-trifluoro-3,3-dimethyl-isoquinoline, 4,4-difluoro-3,3-dimethyl-1-(6-methylpyrazolo[1,5-a]pyridin-3-yl) isoquinoline, 4,4-difluoro-3,3-dimethyl-1-(7-methylpyrazolo[1,5-a]pyridin-3-1-(6-chloro-7-methyl-pyrazolo[1,5-a]pyridin-3-yl)-4,4-difluoro-3,3-yl) isoquinoline, dimethyl-isoquinoline (these compounds may be prepared from the methods described in WO2017/025510); 1-(4,5-dimethylbenzimidazol-1-yl)-4,4,5-trifluoro-3,3-dimethyl-isoquinoline, 1-(4,5-dimethylbenzimidazol-1-yl)-4,4-difluoro-3,3-dimethyl-isoquinoline, 6-chloro-4,4-difluoro-3,3-dimethyl-1-(4-methylbenzimidazol-1-yl) isoquinoline, 4,4-difluoro-1-(5-fluoro-4-methyl-benzimidazol-1-yl)-3,3-dimethyl-isoquinoline, 3-(4,4-difluoro-3,3-dimethyl-1-isoquinolyl)-7,8-dihydro-6H-cyclopenta[e]benzimidazole (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, N-ethyl-2-methyl-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, 3-ethyl-1-methoxy-1-[[4-[5-(trifluoromethyl)-1,2,4-oxadiazol-3-yl]phenyl]methyl]urea, N-[[4-[5-(trifluoromethyl)-1,2,4-oxadiazol-3-yl]phenyl]methyl]propanamide, 4,4-dimethyl-2-[[4-[5-(trifluoromethyl)-1,2,4-oxadiazol-3-yl]phenyl]methyl]isoxazolidin-3-one, 5,5-dimethyl-2-[[4-[5-(trifluoromethyl)-1,2,4-oxadiazol-3-yl]phenyl]methyl]isoxazolidin-3-one, ethyl 1-[[4-[5-(trifluoromethyl)-1,2,4-oxadiazol-3-yl]phenyl]methyl]pyrazole-4-carboxylate, N,N-dimethyl-1-[[4-[5-(trifluoromethyl)-1,2,4-oxadiazol-3-yl]phenyl]methyl]-1,2,4-triazol-3-amine (these compounds may be prepared from the methods described in WO 2017/055473, WO 2017/055469, WO 2017/093348 and WO 2017/118689); 2-[6-(4-chlorophenoxy)-2-(trifluoromethyl)-3-pyridyl]-1-(1,2,4-triazol-1-yl) propan-2-ol (this compound may be prepared from the methods described in WO 2017/029179); 2-[6-(4-bromophenoxy)-2-(trifluoromethyl)-3-pyridyl]-1-(1,2,4-triazol-1-yl) propan-2-ol (this compound may be prepared from the methods described in WO 2017/029179); 3-[2-(1-chlorocyclopropyl)-3-(2-fluorophenyl)-2-hydroxy-propyl]imidazole-4-carbonitrile (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile (this compound may be prepared from the methods described in WO 2016/156290); (4-phenoxyphenyl)methyl 2-amino-6-methyl-pyridine-3-carboxylate (this compound may be prepared from the methods described in WO 2014/006945); 2,6-Dimethyl-1H,5H-[1,4]dithiino[2,3-c: 5,6-c′]dipyrrole-1,3,5,7 (2H,6H)-tetrone (this compound may be prepared from the methods described in WO 2011/138281) N-methyl-4-[5-(trifluoromethyl)-1,2,4-oxadiazol-3-yl]benzenecarbothioamide; N-methyl-4-[5-(trifluoromethyl)-1,2,4-oxadiazol-3-yl]benzamide; (Z,2E)-5-[1-(2,4-dichlorophenyl)pyrazol-3-yl]oxy-2-methoxyimino-N,3-dimethyl-pent-3-enamide (this compound may be prepared from the methods described in WO 2018/153707); N′-(2-chloro-5-methyl-4-phenoxy-phenyl)-N-ethyl-N-methyl-formamidine; N′-[2-chloro-4-(2-fluorophenoxy)-5-methyl-phenyl]-N-ethyl-N-methyl-formamidine (this compound may be prepared from the methods described in WO 2016/202742); 2-(difluoromethyl)-N-[(3S)-3-ethyl-1,1-dimethyl-indan-4-yl]pyridine-3-carboxamide (this compound may be prepared from the methods described in WO 2014/095675); (5-methyl-2-pyridyl)-[4-[5-(trifluoromethyl)-1,2,4-oxadiazol-3-yl]phenyl]methanone, (3-methylisoxazol-5-yl)-[4-[5-(trifluoromethyl)-1,2,4-oxadiazol-3-yl]phenyl]methanone (these compounds may be prepared from the methods described in WO 2017/220485); 2-oxo-N-propyl-2-[4-[5-(trifluoromethyl)-1,2,4-oxadiazol-3-yl]phenyl]acetamide (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate (this compound may be prepared from the methods described in WO 2018/158365); 2,2-difluoro-N-methyl-2-[4-[5-(trifluoromethyl)-1,2,4-oxadiazol-3-yl]phenyl]acetamide, N-[(E)-methoxyiminomethyl]-4-[5-(trifluoromethyl)-1,2,4-oxadiazol-3-yl]benzamide, N-[(Z)-methoxyiminomethyl]-4-[5-(trifluoromethyl)-1,2,4-oxadiazol-3-yl]benzamide, N-[N-methoxy-C-methyl-carbonimidoyl]-4-[5-(trifluoromethyl)-1,2,4-oxadiazol-3-yl]benzamide (these compounds may be prepared from the methods described in WO 2018/202428).

In particular, the following fungicidal mixing partners are preferred:
a strobilurin fungicide selected from the group consisting of azoxystrobin, dimoxystrobin, fluoxastrobin, kresoxim-methyl, metominostrobin, orysastrobin, picoxystrobin, pyraclostrobin, trifloxystrobin;
an azole fungicide selected from the group consisting of azaconazole, bromuconazole, cyproconazole, difenoconazole, diniconazole, diniconazole-M, epoxiconazole, fenbuconazole, fluquinconazole, flusilazole, flutriafol, hexaconazole, imazalil, imibenconazole, ipconazole, metconazole, myclobutanil, oxpoconazole, pefurazoate, penconazole, prochloraz, propiconazole, prothioconazole, simeconazole, tebuconazole, tetraconazole, triadimefon, triadimenol, triflumizole, triticonazole, diclobutrazol, etaconazole, furconazole, furconazole-cis and quinconazole;
a morpholine fungicide selected from the group consisting of aldimorph, dodemorph, fenpropimorph, tridemorph, fenpropidin, spiroxamine and piperalin;
an anilino-pyrimidine fungicide selected from the group consisting of cyprodinil, mepanipyrim and pyrimethanil; and/or
a fungicide selected from the group consisting of benalaxyl, benalaxyl-M, benomyl, bitertanol, boscalid, captan, carboxin, carpropamid, chlorothalonil, copper, cyazofamid, cymoxanil, diethofencarb, dithianon, famoxadone, fenamidone, fenhexamide, fenoxycarb, fenpiclonil, fluazinam, fludioxonil, flutolanil, folpet, guazatine, hymexazole, iprodione, lufenuron, mancozeb, metalaxyl, mefenoxam, metrafenone, nuarimol, paclobutrazol, pencycuron, penthiopyrad, procymidone, proquinazid, pyroquilon, quinoxyfen, silthiofam, sulfur, thiabendazole, thiram, triazoxide, tricyclazole, isopyrazam, sedaxane, fluxapyroxad, solatenol and 3-(difluoromethyl)-N-methoxy-1-methyl-N-[1-methyl-2-(2,4,6-trichlorophenyl)ethyl]pyrazole-4-carboxamide.

Even more preferably, the polymorph of the invention is combined with a strobilurin or azole.

Whilst compositions comprising the polymorph of the invention and another fungicide are explicitly disclosed above, the skilled person will appreciate that the invention extends to three-way, and further multiple combinations comprising the above two-way mixtures.

For the avoidance of doubt, even if not explicitly stated above, the mixing partners of may also be in the form of any suitable agrochemically acceptable ester or salt, as mentioned e.g. in The Pesticide Manual, Nineteenth Eidtion, British Crop Protection Council 2021.

The present invention will now be described by way of the following non-limiting examples and figures, wherein:

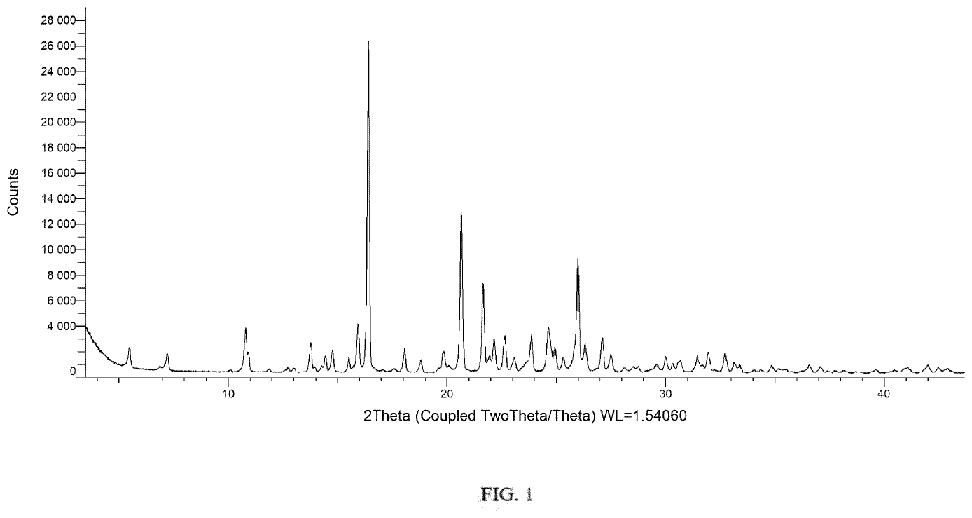

FIG. 1 shows the powder X-ray diffraction pattern of the crystalline polymorph (B form) according to the present invention.

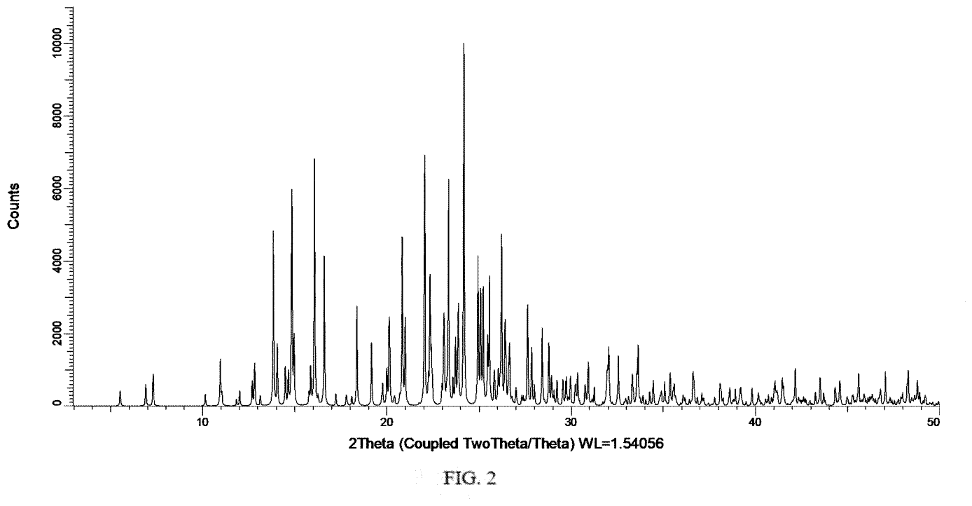

FIG. 2 shows the X-ray diffraction pattern calculated from the unit cell parameters according to Table 2, of the crystalline polymorph (B form) according to the present invention.

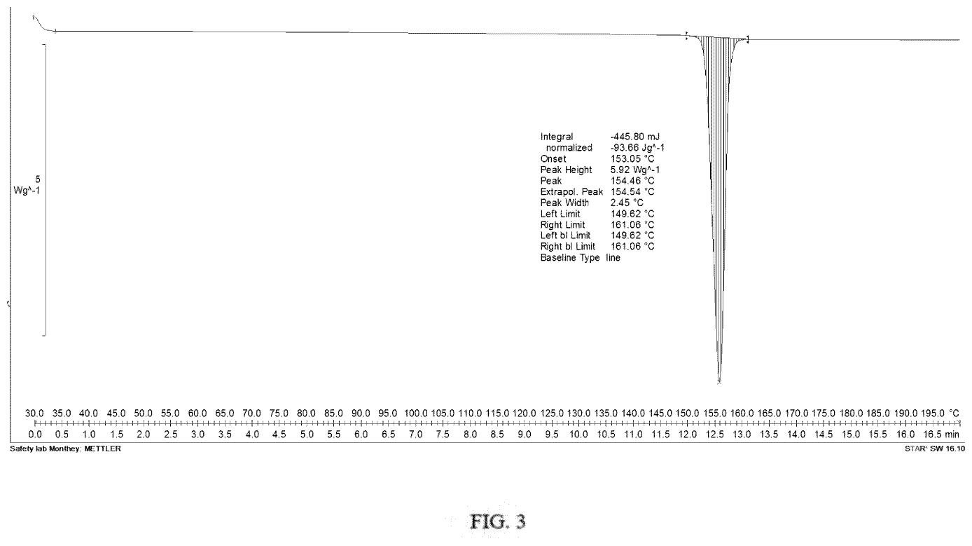

FIG. 3 shows a DSC trace of the crystalline polymorph (B form) according to the present invention.

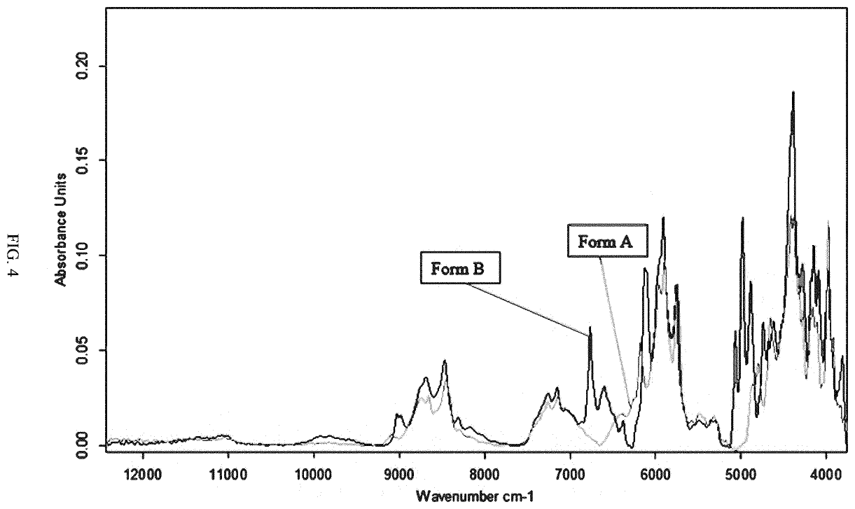

FIG. 4 shows near infra-red spectroscopy traces of the crystalline polymorph (B form) according to the present invention, and of the crystalline polymorph (A form) according to the prior art.

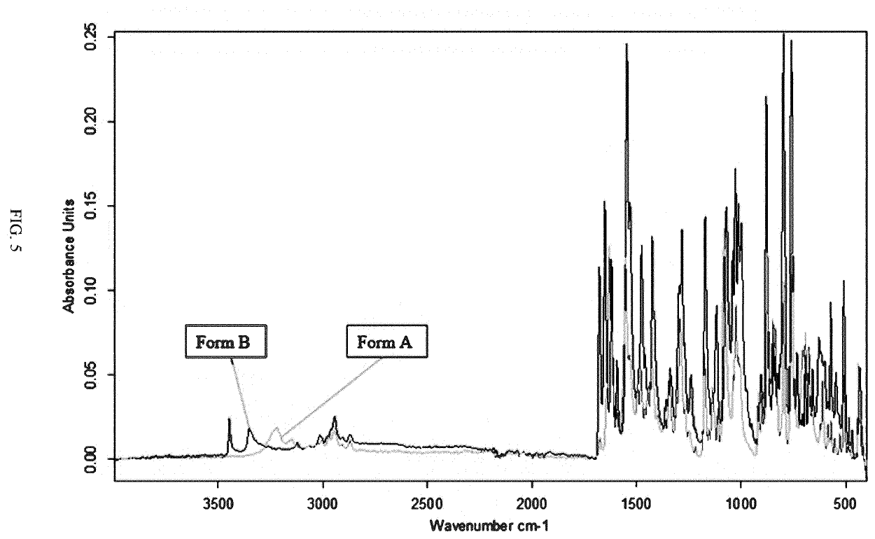

FIG. 5 shows mid infra-red spectroscopy traces of the crystalline polymorph (B form) according to the present invention, and of the crystalline polymorph (A form) according to the prior art.

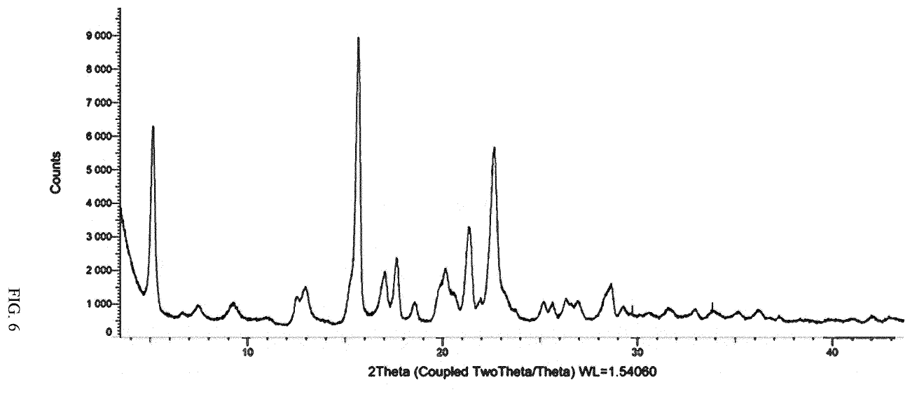

FIG. 6 shows the powder X-ray diffraction pattern of the crystalline polymorph (A form) according to the prior art.

### EXAMPLES

### 1. Preparation of Polymorph

The technical compound of formula I used in the preparation of the polymorph according to the present invention is a racemic mixture of the compound of formula I (3-difluoromethyl-1-methyl-1H-pyrazole-4-carboxylic acid (9-dichloromethylene-1,2,3,4-tetrahydro-1,4-methano-naphthalen-5-yl)-amide) with a purity of 99.0%, prepared as described in Example P11 of WO 2012/101139.

Xylene (580 g) was added to a 1000 mL jacketed crystallization vessel and heated to 40 °C, with the agitation speed set at 100 rpm. The technical compound (30 g) of formula I was added slowly, and the mixture was stirred at 40 °C for 30 min until only a small amount of solids remained (a sign that the saturation was achieved). The mixture was then heated to 45 °C to ensure complete dissolution, and was filtered hot (at 45 °C) through a preheated funnel equipped with a paper filter (Type 1) into 4 preheated glass bottles, each with a magnetic stirrer. The stirred solution was allowed to cool slowly to room temperature (20 °C) with stirring, more particularly with mild (magnetic) stirring, and the obtained suspension was kept at room temperature for 4 weeks with stirring, more particularly with mild (magnetic) stirring. The suspension was filtered, and solids were analysed to confirm the formation of the desired polymorph (B form) according to the invention.

### 2. Analysis of Polymorphs

After preparation by the methods detailed above, the samples were subject to analysis by powder X-ray diffraction, and/or single crystal X-ray diffraction, and/or differential scanning calorimetry (DSC), and/or near infra-red spectroscopy, and/or mid infra-red spectroscopy.

Powder X-ray diffraction analysis of solid material was carried out using a Bruker D2 Phaser powder diffractometer at room temperature (20 °C) and at relative humidities above 40%. Samples were mounted in a standard PMMA sample holders and the samples flattened. The sample holder was rotated, and X-rays were collected from 3.5 to 44° 2θ with a step size of 0.02° and incident x-rays with wavelength of 1.5406 Å. The powder X-ray diffraction pattern of the crystalline polymorph (B form) according to the invention is shown in FIG. 1, and the powder X-ray diffraction pattern of the crystalline polymorph (A form) according to the prior art is shown in FIG. 6.

Single crystal intensity data was collected on an Rigaku Supernova diffractometer using Cu Kα radiation (λ=1.54056 Å) with a graphite monochromator. The crystal was mounted in NVH oil at −173 °C for data collection. The data was solved using the CRYSTALS software package, and the results are gathered in Table 2 (unit cell parameters). The X-ray diffraction pattern calculated from the unit cell parameters of the crystalline polymorph (B form) is shown in FIG. 2.

DSC was carried out using a Mettler Toledo DSC1, using standard 40 μL aluminium sample holders with pierced lids (to allow the escape of any gas formed during the heating of the sample), heating from 30 to 200 °C at a rate of 10 °C/minute. The DSC trace of the crystalline polymorph (B form) according to the invention is shown in FIG. 3.

Near infra-red spectroscopy analyses were performed on a Bruker MPA1 spectrometer using an integrating sphere from 3,500 to 12,500 cm⁻¹ at room temperature (25 °C). The sample (as received) was transferred to a glass vial. Sufficient sample was used to ensure all the light source was bounced back onto the detector. The near infra-red spectroscopy traces of the crystalline polymorph (B form) according to the invention and of the crystalline polymorph (A form) according to the prior art are shown in FIG. 4.

Mid infra-red spectroscopy analyses were performed on a Bruker Alpha II spectrometer from 400 to 4,000 cm⁻¹, without any sample preparation. The Alpha II (FTIR) has an anvil head that presses the sample onto the diamond window for analysis. The mid infra-red spectroscopy traces of the crystalline polymorph (B form) according to the invention and of the crystalline polymorph (A form) according to the prior art are shown in FIG. 5.

### 3. Experiments

In order to demonstrate the advantage of the crystalline polymorph according to the present invention, the following experiments were performed.

### 3.1. Formation of the Seeds

Solid seeds B were prepared according to the above-mentioned polymorph preparation of the invention (see item “1. Preparation of Polymorph”).

Solid seeds A were prepared according to WO 2012/101139 (Example P11), wherein the powder X-ray diffraction pattern is shown in FIG. 6, and is related to the anhydrous form 1 described in WO 2015/128322. Said solid seeds A (i.e. anhydrous form 1) can also be prepared according to WO 2011/131544 (step f of Preparatory examples), WO 2011/131545 (step g of Preparatory examples), WO 2011/131546 (step d of Preparatory examples), or US 2012/136162 (Example P6 or Example P7).

### 3.2. Seeded Process

Xylene (774 g) and the technical compound (125 g) of formula I were added to a 1000 ml jacketed vessel equipped with an anchor stirrer (agitation speed: 150 rpm), and heated to 75 °C until all solids dissolved.

After the solution was allowed to cool down to 50 °C (seeding temperature), solid seeds B (2.0 g) or solid seeds A (2.0 g) were added. The suspension was kept at 50 °C for 60 minutes, and then cooled down to 0 °C over 20 hours (linear cooling ramp from 50 °C to 0 °C for 20 hours). The suspension was discharged into the filtration unit, for the specific cake resistance measurements.

### 3.3. Filtration Experiments

In a first part, the suspension was charged on the filtration unit, equipped with a 20 μm polytetrafluoroethylene filter with a diameter of 5 cm. The equipment was put under pressure (+0.5 bar gauge (barg)), and filtration was started. Once the surface of the cake was exposed, the filtration was terminated, and the filtrates were recharged in the vessel and cooled down to 0 °C. In the second part, the filtration was subsequently repeated at a pressure of +0.5 barg. The final product was dried at 57 °C under vacuum (1-10 mbar), to give the polymorph B or A.

In terms of filtration, the initial run establishes the cake on the filter and allows assessment of the filter media (cloth) resistance. The subsequent run allows calculation of the specific cake resistance. The data required are the pressure, the filtrate liquid viscosity (which is 0.75 mPa's), filtrate quantity collected with time, and the final cake height after filtration.

The below Table 3 and Table 4 summarise the results obtained for the second part of each experiment from which the specific cake resistance is calculated. More particularly, Table 3 (Example 1) is according to the present invention (crystals of Form B), wherein the cake height is 0.100 m, and Table 4 (Example 2) is a comparative example (crystals of Form A), wherein the cake height is 0.199 m. All the experiments are performed in the same way, apart from the solid used (B or A).

TABLE 3
Example 1

| Filtration time (s) | Filtrate quantity [10⁻⁵ m³] |
|---|---|
| 0 | 0 |
| 4 | 0.120 |
| 8 | 0.335 |
| 12 | 0.743 |
| 16 | 0.946 |
| 20 | 1.354 |
| 24 | 1.540 |
| 28 | 1.540 |
| 32 | 1.931 |
| 36 | 2.190 |
| 40 | 2.515 |
| 44 | 2.704 |
| 48 | 3.085 |
| 52 | 3.275 |
| 56 | 3.275 |
| 60 | 3.845 |
| 64 | 3.845 |
| 68 | 4.226 |
| 72 | 4.403 |
| 76 | 4.777 |
| 80 | 4.965 |
| 84 | 5.336 |
| 88 | 5.521 |
| 92 | 5.894 |
| 96 | 6.081 |
| 100 | 6.081 |
| 104 | 6.636 |
| 108 | 6.636 |
| 112 | 6.994 |
| 116 | 7.151 |
| 120 | 7.252 |
| 124 | 7.257 |

TABLE 4
Example 2

| Filtration time (s) | Filtrate quantity [10⁻⁵ m³] |
|---|---|
| 0 | 0 |
| 4 | 0.0427 |
| 8 | 0.0587 |
| 12 | 0.0743 |
| 16 | 0.0916 |
| 20 | 0.1024 |
| 24 | 0.1024 |
| 28 | 0.1243 |
| 32 | 0.1337 |
| 36 | 0.1534 |
| 40 | 0.1622 |
| 44 | 0.1809 |
| 48 | 0.1899 |
| 52 | 0.1899 |
| 56 | 0.2082 |
| 60 | 0.2208 |
| 64 | 0.2357 |
| 68 | 0.2470 |
| 72 | 0.2618 |
| 76 | 0.2713 |
| 80 | 0.2884 |
| 84 | 0.3008 |
| 88 | 0.3008 |
| 92 | 0.3239 |
| 96 | 0.3239 |
| 100 | 0.3390 |
| 104 | 0.3501 |
| 108 | 0.3649 |
| 112 | 0.3739 |
| 116 | 0.3912 |
| 120 | 0.3999 |
| 124 | 0.4168 |

The cloth resistance for both Examples 1 and 2 is 1.14×10¹⁰ m⁻¹.

The specific cake resistance r_{cake} can be calculated using the following equation:

$$\dot{V} = \frac{A \cdot Δ p}{μ \cdot [ {R}_{cloth} + {r}_{c a k e} \cdot {h}_{c} ]}$$

wherein: R_{cloth}=cloth (filter) resistance expressed in [m⁻¹]; A=area of the filter [m²]; Δp=differential pressure [Pa]; μ=dynamic viscosity [Pa·s]; r_{cake}=specific cake resistance [m⁻²]; h_{c}=height of the cake [m]; \dot{V}=volumetric flowrate of filtrate [m³·s⁻¹].

The compressibility data as described on the equations above is summarized in Table 5.

TABLE 5

| Example | r_{cake} [m⁻²] |
|---|---|
| 1 | 9.4 × 10¹⁰ |
| 2 | 211 × 10¹⁰ |

It stems from the results of Table 5 that the cake obtained from Example 1 according to the present invention has a specific cake resistance at least twenty times lower than the cake obtained from Example 2, so that the filtration with the polymorph B is faster than with the polymorph A. This advantage is a significant improvement during the filtration step(s) of the compound of formula I according to the present invention, especially in reducing the time of filtration.

Although the invention has been described with reference to preferred embodiments and examples thereof, the scope of the present invention is not limited only to those described embodiments. As will be apparent to persons skilled in the art, modifications and adaptations to the above-described invention can be made without departing from the spirit and scope of the invention, which is defined and circumscribed by the appended claims. All publications cited herein are hereby incorporated by reference in their entirety for all purposes to the same extent as if each individual publication were specifically and individually indicated to be so incorporated by reference.

1. A crystalline polymorph of the compound of formula I 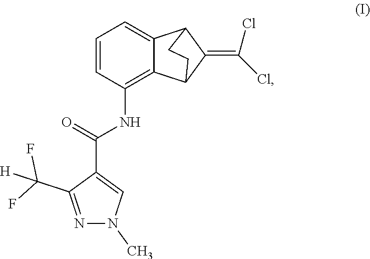 [SMILES: Cn1cc(C(=O)Nc2cccc3c2C2CCC3C2=C(Cl)Cl)c(C(F)F)n1]
which has a powder X-ray diffraction pattern comprising at least three 2θ angle values selected from the group consisting of 7.2±0.2°, 10.8±0.2°, 13.8±0.2°, 14.4±0.2°, 14.8±0.2°, 15.9±0.2°, 16.4±0.2°, 19.9±0.2°, 20.7±0.2°, 22.2±0.2°, 22.6±0.2°, 23.9±0.2°, 24.6±0.2°, 26.0±0.2°, 26.3±0.2°, 27.5±0.2°, 30.0±0.2°, 32.0±0.2°, and 32.7±0.2°.

2. The crystalline polymorph according to claim 1, characterized in that the powder X-ray diffraction pattern comprises at least six 2θ angle values selected from the group consisting of 7.2±0.2°, 10.8±0.2°, 13.8±0.2°, 14.4±0.2°, 14.8±0.2°, 15.9±0.2°, 16.4±0.2°, 19.9±0.2°, 20.7±0.2°, 22.2±0.2°, 22.6±0.2°, 23.9±0.2°, 24.6±0.2°, 26.0±0.2°, 26.3±0.2°, 27.5±0.2°, 30.0±0.2°, 32.0±0.2°, and 32.7±0.2°.

3. The crystalline polymorph according to claim 1, characterized in that the powder X-ray diffraction pattern comprises at least the following 2θ angle values: 7.2±0.2°, 10.8±0.2°, 13.8±0.2°, 14.4±0.2°, 14.8±0.2°, 16.4±0.2°, 20.7±0.2°, 22.2±0.2°, 24.6±0.2°, and 26.0±0.2°.

4. The crystalline polymorph according to claim 1, characterized in that it has the following lattice parameters: a=8.0±0.1 Å, b=14.0 Å±0.1 Å, c=17.0 Å±0.1 Å, α=70.2±0.2°, β=84.1±0.2°, γ=74.4±0.2°, and volume=1724±20 Å³.

5. The crystalline polymorph according to claim 1, characterized in that it has a melting point ranging from 153 °C to 156 °C (peak position), and preferably ranging from 154 °C to 155 °C (peak position).

6. An agrochemical composition comprising the crystalline polymorph as claimed in claim 1, and at least one agriculturally acceptable carrier or diluent.

7. The composition according to claim 6, characterized in that it comprises at least one further fungicide.

8. The composition according to claim 6, characterized in that the further fungicide is a strobilurin or an azole.

9. The composition according to claim 6, characterized in that it further comprises at least one insecticide and/or at least one nematicide.

10. A method of preventing or controlling fungal infection on plants or plant propagation material comprising treating the plant or plant propagation material with the agrochemical composition as claimed in claim 6.
